(19) United States
(12) Patent Application Publication
(10) Pub. No.: US 2013/0095127 A1
(43) Pub. Date: Apr. 18, 2013

(54) METHODS OF INHIBITING INFLAMMATION AND INFLAMMATORY DISEASES USING Gal-3BP (BTBD17B, LGALS3BP, GALECTIN-3 BINDING PROTEIN, MAC-2 BINDING PROTEIN)

(71) Applicant: LA JOLLA INSTITUTE FOR ALLERGY AND IMMU, La Jolla, CA (US)

(72) Inventors: Klaus Ley, La Jolla, CA (US); Iftach Shaked, La Jolla, CA (US)

(73) Assignee: LA JOLLA INSTITUTE FOR ALLERGY AND IMMUNOLOGY, La Jolla, CA (US)

(21) Appl. No.: 13/627,839

(22) Filed: Sep. 26, 2012

Related U.S. Application Data
(63) Continuation of application No. PCT/US2010/053435, filed on Oct. 20, 2010.
(60) Provisional application No. 61/357,839, filed on Jun. 23, 2010.
(60) Provisional application No. 61/318,146, filed on Mar. 26, 2010.

(51) Int. Cl. A61K 38/17; A61K 45/06; C07K 14/435

(52) U.S. Cl. CPC A61K 38/1709; C07K 14/435; A61K 45/06
USPC 424/185.1

(57) ABSTRACT

The invention provides Galectin-3 binding protein (Gal-3BP, BTBD17B) polypeptides, Gal-3BP compositions, and methods of use, and uses, for example, in treatment, diagnostic, detection and prognostic methods, such as treatment of an undesirable or aberrant immune response, immune disorder, inflammatory response, or inflammation, and treatment of an autoimmune response, disorder or disease.

### RELATED APPLICATIONS

This application claims the benefit of priority of application Ser. No. 61/318,146, filed Mar. 26, 2010, and application Ser. No. 61/357,839, filed Jun. 23, 2010, each of which applications are expressly incorporated by reference herein in their entirety.

### STATEMENT AS TO FEDERALLY SPONSORED RESEARCH

This invention was made with Government support under Grant No. HL058108 awarded by the National Institutes of Health. The Government has certain rights in this invention.

### INTRODUCTION

We have now discovered parts of the Gal-3BP (also known as BTBD17B, LGALS3BP, Galectin-3 Binding Protein, and Mac-2 Binding Protein) signaling pathway (requires NFAT) and IL-2 as a significant downstream effector cytokine. This is based on studies with isolated mouse and human macrophages, cell lines and Lgals3bp−/− mice.

### SUMMARY

The invention provides methods of decreasing, reducing, inhibiting, suppressing, limiting or controlling an undesirable or aberrant immune response, immune disorder, inflammatory response, or inflammation. In one embodiment, a method includes administering a Galectin-3 binding protein (Gal-3BP, BTBD17B) polypeptide to a subject in an amount that increases Gal-3BP polypeptide in the subject thereby decreasing, reducing, inhibiting, suppressing, limiting or controlling an undesirable or aberrant immune response, immune disorder, inflammatory response, or inflammation in the subject.

The invention also provides methods of decreasing, reducing, inhibiting, suppressing, limiting or controlling an autoimmune response, disorder or disease. In one embodiment, a method includes administering a Galectin-3 binding protein (Gal-3BP) polypeptide to a subject in an amount to decrease, reduce, inhibit, suppress, limit or control an autoimmune response, disorder or disease in the subject.

In various non-limiting aspects of the invention methods, the treatment decreases, reduces, inhibits, suppresses, limits or controls an adverse symptom of the undesirable or aberrant immune response, immune disorder, inflammatory response, or inflammation, or an adverse symptom of the autoimmune response, disorder or disease.

In various additional non-limiting aspects of the invention methods, the adverse symptom of the undesirable or aberrant immune response, immune disorder, inflammatory response, or inflammation, or an adverse symptom of the autoimmune response, disorder or disease is swelling, pain, rash, headache, fever, nausea, diarrhea, bloat, lethargy, skeletal joint stiffness, or tissue or cell damage. In various further non-limiting aspects of the invention methods, the adverse symptom of the undesirable or aberrant immune response, immune disorder, inflammatory response, or inflammation, or an adverse symptom of the autoimmune response, disorder or disease is in an epidermal or mucosal tissue, gut, bowel, pancreas, thymus, liver, kidney, muscle, central or peripheral nerves, spleen, skin, or a skeletal joint (e.g., knee, ankle, hip, shoulder, wrist, finger, toe, or elbow).

Invention methods can be used to treat a chronic or acute adverse symptom of the undesirable or aberrant immune response, immune disorder, inflammatory response, or inflammation, or autoimmune response, disorder or disease, or an adverse symptom thereof. Invention methods can also be used to treat an antibody or cell mediated undesirable or aberrant immune response, immune disorder, inflammatory response, or inflammation, or autoimmune response, disorder or disease.

Invention methods and uses include methods and uses in which a Galectin-3 binding protein (BTBD17B) polypeptide or fragment thereof stimulates, promotes, increases or induces interleukin-2 (IL-2) production by or secretion from macrophages or dendritic cells. In a particular aspect, the interleukin-2 (IL-2) stimulates, promotes, increases or induces survival, proliferation or differentiation of regulatory T cells (T regs).

Invention methods and uses include methods and uses in which a Galectin-3 binding protein (BTBD17B) polypeptide or fragment thereof stimulates, promotes, increases or induces nuclear factor of activated T cells (NFAT) activation. In a particular aspect, NFAT activation is indicated by stimulated, promoted, increased or induced macrophage microRNA expression, has-miR-21, has-miR-23b, has-miR-24, has-miR-27a or has-miR-125a-5p.

Invention methods and uses include methods and uses in which a Galectin-3 binding protein (BTBD17B) polypeptide or fragment thereof decreases, reduces or inhibits a pro-inflammatory cytokine (e.g., tumor necrosis factor alpha, TNF-α, or interleukin-6, IL-6) or interleukin-10 (IL-10) production or secretion by splenocytes or macrophages.

The invention additionally provides methods and uses of stimulating, promoting, increasing or inducing interleukin-2 (IL-2) production by or secretion from macrophages or dendritic cells. In one embodiment, a method includes administering Galectin-3 binding protein (BTBD17B) polypeptide or fragment thereof to a subject in an amount that stimulates, promotes, increases or induces interleukin-2 (IL-2) production by or secretion from macrophages or dendritic cells.

The invention further provides methods and uses of decreasing, reducing or inhibiting TNF-alpha or IL-10 or IL-6 production or secretion by splenocytes or macrophages. In one embodiment, a method includes administering Galectin-3 binding protein (BTBD17B) polypeptide or fragment thereof to a subject in an amount that decreases, reduces or inhibits TNF-alpha or IL-10 or IL-6 production or secretion by splenocytes or macrophages.

Methods and uses of the invention moreover include increasing Gal-3BP polypeptide to an amount greater than prior to administration. In particular embodiments, Gal-3BP polypeptide increases to an amount in the subject of greater than 2 ug/ml in blood plasma, increases to an amount greater than 5 ug/ml in blood plasma, greater than 10 ug/ml in blood plasma, greater than 15 ug/ml in blood plasma, or greater than 20 ug/ml in blood plasma. Methods and uses of the invention also include increasing Gal-3BP polypeptide to an amount greater than prior to administration for a period of time greater than 12, 24, 36, 48, 72 hours, or 3, 4, 5, 6, 7, 8, 9, 10, 12, 14 days, weeks or months.

The invention moreover provides Gal-3BP polypeptide combinations. In one embodiment, a combination includes Gal-3BP polypeptide and a second drug or agent, such as an anti-inflammatory drug or agent. Such compositions are useful in the methods and uses of the invention.

Gal-3BP polypeptides according to the invention include mammalian forms, such as human. Additional forms include non-human primates (e.g., Pan troglodytes), dogs (e.g., Canis lupus familiaris), cattle (Bos Taurus), and rodents (e.g., Mus musculus and Rattus norvegicus).

Gal-3BP polypeptide according to the invention include full length Gal-3BP polypeptide, as well as modified forms of Gal-3BP polypeptide, such as fragments and chimeras and fusions. Non-limiting exemplary polypeptide fragments of Gal-3BP polypeptide include all or a portion of residues 24-124 (SRCR domain); residues 153-221 (BTB domain); or residues 260-360 (BACK domain).

The invention compositions, methods and uses therefore include full length Gal-3BP polypeptide and modified forms, such as subsequences of full length Galectin-3 binding protein (Gal-3BP) polypeptide, that have a function or activity of unmodified (e.g., full length) Galectin-3 binding protein, such as an activity or function set forth herein. In various embodiments, a modified form (e.g., a subsequence) includes a modified Galectin-3 binding protein that decreases, reduces, inhibits, suppresses, limits or controls an undesirable or aberrant immune response, immune disorder, inflammatory response, or inflammation, or an autoimmune response, disorder or disease. In an additional embodiment, a composition, method or use includes a modified Galectin-3 binding protein (e.g., a subsequence or fragment of full length Galectin-3 binding protein) that stimulates, promotes, increases or induces interleukin-2 (IL-2) production by or secretion from macrophages or dendritic cells, that stimulates, promotes, increases or induces nuclear factor of activated T cells (NFAT) activation, or that decreases, reduces or inhibits a pro-inflammatory cytokine (e.g., TNF-alpha or IL-6) or interleukin-10 (IL-10) production or secretion by splenocytes or macrophages.

In particular aspects, a Galectin-3 binding protein (Gal-3BP) subsequence consists of Gal-3BP residues 24-124 (SRCR domain); residues 153-221 (BTB domain); residues 260-360 (BACK domain), or a subsequence of Gal-3BP residues 24-124 (SRCR domain); residues 153-221 (BTB domain); residues 260-360 (BACK domain), or is about 5-10, 10-20, 20-50, 50-75 or 50-100 amino acids in length and includes all or a portion of Gal-3BP residues 24-124 (SRCR domain); residues 153-221 (BTB domain); or residues 260-360 (BACK domain). Non-limiting additional subsequences of Galectin-3 binding protein (Gal-3BP) polypeptide are about 5-10, 10-20, 20-50, 50-100, 100-150, 150-200, 200-250, 250-300, 300-350, 350-400, 400-500, 500-600 or more amino acids in length, and less than full length BTBD17B polypeptide sequence.

Such full length Gal-3BP polypeptide, and modified forms of Galectin-3 binding protein (Gal-3BP) (e.g., a subsequences), include isolated and purified forms, as well as combination compositions and pharmaceutical forms.

Additionally provided are a Gal-3BP polypeptide (e.g., full length) or modified form, compositions and pharmaceutical compositions, and combinations, such compositions and combinations used in the methods or uses of the invention. In one embodiment, a method includes a Gal-3BP polypeptide (e.g., full length) or modified form composition, and optionally a pharmaceutically acceptable carrier (e.g., such as saline). In another embodiment, a method includes a modified Galectin-3 binding protein (e.g., a subsequence or fragment of full length Galectin-3 binding protein) composition that decreases, reduces, inhibits, suppresses, limits or controls an undesirable or aberrant immune response, immune disorder, inflammatory response, or inflammation, or an autoimmune response, disorder or disease. In an additional embodiment, a method includes a modified Galectin-3 binding protein (e.g., a subsequence or fragment of full length Galectin-3 binding protein) composition that stimulates, promotes, increases or induces interleukin-2 (IL-2) production by or secretion from macrophages or dendritic cells, that stimulates, promotes, increases or induces nuclear factor of activated T cells (NFAT) activation, or that decreases, reduces or inhibits a pro-inflammatory cytokine (e.g., TNF-alpha or IL-6) or interleukin-10 (IL-10) production or secretion by splenocytes or macrophages.

Invention compositions, methods and uses can include Gal-3BP polypeptide (or a modified form) or a composition thereof in any amount. Non-limiting amounts include Gal-3BP polypeptide at a concentration of about 1 mg/ml, or in a range of about 100 μg/ml to 1,000 mg/ml, in an amount of 10-1,000 milligrams, or in an amount of between about 1-100 milligrams.

Candidate subjects for methods and uses of the invention, including treatment methods and uses have or are at increased risk of an acute or chronic an undesirable or aberrant immune response, immune disorder, inflammatory response, or inflammation, or an autoimmune response, disorder or disease. Candidate subjects for methods of the invention include human subjects in need of treatment for an undesirable or aberrant immune response, immune disorder, inflammatory response, or inflammation, or an autoimmune response, disorder or disease.

Subjects can optionally be excluded. For example, a subject that has been diagnosed for an adverse cardiovascular event or cardiovascular disease; a subject that has been diagnosed for coronary artery disease, peripheral artery disease, cerebrovascular disease, or renal artery disease; a subject that has had a stroke, myocardial infarction (heart attack), ischemic heart failure, transient ischemic attack or brain trauma; a subject in need of treatment or therapy for artherosclerotic plaque formation, elevated blood cholesterol or a cardiovascular disease, or a subject that has been diagnosed for cancer, has been treated for cancer, or is in remission from cancer, can be excluded from a method or use.

### DESCRIPTION OF THE DRAWINGS

FIGS. 1A-1C show that Gal-3BP induces IL-2 secretion in human macrophages A) Only Gal-3BP induced production of IL-2 as measured by standard ELISA (***P=0.0003). B) Gal-3BP concentration dependently induces IL-2 production in human macrophages. Human blood monocytes were cultured as described in Example 2 (*P=0.05, ***P<0.0001).

These macrophages were incubated with Gal-3BP (0.1,1 and 10 μg/ml) for 6 hours and IL-2 was measured by ELISA. C) Gal-3BP but not LPS induces IL-2 production in human macrophages.

FIGS. 2A-2D show that Gal-3BP induces a unique macrophage phenotype. Only Gal-3BP induced A) moderate IL-10 (typical of M2 and Mreg); B) production of IL-2; C) no IL-12 (typical of M1); and D) little IL-6 (typical of M1). P values are indicated.

FIGS. 3A-3B show that M1 macrophages produce Gal-3BP. A) Gal3BP mRNA expression was measured by quantitative RT-PCR, expressed relative to H18S (**P=0.0056 by paired t test, n=3 donors); B) Gal-3BP protein production was measured using ELISA (***P=0.0008). Only M1 macrophages induce Gal-3BP mRNA expression (A) and secrete significant amounts of Gal-3BP (B), although levels in the supernatant remain about 10 times lower than in blood.

FIG. 4 shows that Gal-3BP triggers Ca²⁺ mobilization. Human blood monocytes were cultured as in the previous figures to produce M0 macrophages, incubated with 1 uM fluo-4 for 30 minutes to quantify intracellular free Ca²⁺ concentrations, and then triggered with Gal-3BP with or without the PLC-γ inhibitor U73122. Thapsigargin served as positive control. Ca²⁺ mobilization was assayed by fluorescence microscopy. Each line is a measurement from a single cell.

FIG. 5 shows that Gal-3BP induces NFAT translocation to nucleus in human blood monocytes. Samples were stained with DAPI (nuclear stain) and an antibody to NFAT. Nuclear translocation was assessed by immunofluorescence microscopy. (NT=not treated)

FIGS. 6A-6B show that NFAT is required for Gal-3BP-induced IL-2 secretion. A) Macrophages incubated with Gal-3BP for the times indicated, which induced secretion of IL-2. IL-2 secretion was partially inhibited by cyclosporine A (P values are 0.04, 0.04, 0.01 and 0.004, left to right respectively). B) Macrophages were alternatively treated with Gal-3BP in the absence or presence of CSA or FK506 inhibitor. FK506 also inhibited IL-2 secretion by Gal-3BP (P values are ***0.0004, **0.006 and *0.034).

FIG. 7 shows that microRNA expression is induced by Gal-3BP. Out of eight microRNAs known to be transcriptional targets of NFAT, five were significantly up-regulated.

FIGS. 8A-8B show that dendritic cells from mice deficient in Gal-3BP fail to produce IL-2. A) IL-2 measured by standard ELISA (P values are *0.02 and ***0.008, respectively); B) BMDCs from Gal-3BP deficient mice produced normal amount of inflammatory cytokines yet fail to produce IL-2. IL-2, TNF-α and IL-6 were measured by ELISA and nitric oxide (NO) was measured using the Griess reagent.

FIG. 9 shows that coupling LPS stimulation with Ca2+ mobilization restores IL-2 production in Gal-3BP deficient mice (CYCAP).

FIG. 10 shows that p65 nuclear translocation is intact in Gal-3BP knock-out mouse dendritic cells.

FIGS. 11A-11D show that Gal-3BP limits LPS-induced inflammatory cytokine production in mouse splenocytes. Production of pro-inflammatory cytokines TNF-α (A) and IL-6 (C), and anti-inflammatory cytokine IL-10 (B) was measured by standard ELISA. (D) Spleen weight and appearance was compared between Gal3BP deficient mice (CYCAP) (n=5) and wild-type mice (WT) (n=5) (P value is 0.0075).

### DETAILED DESCRIPTION

The invention is based, at least in part, on the identification of Galectin-3 binding protein (Gal3-BP, also known as BTBD17B, Mac-2 binding protein or 90K, gene name LGALS3BP in human, also known as CyCAP, MAC-2BP or Ppicap, murine gene name Lgals3bp) as a modulator of immune and inflammatory responses. In particular, as disclosed herein for example, Gal-3BP limits production or expression of pro-inflammatory cytokines, such as TNF-α and interleukin 6 (IL-6). As also disclosed herein, Gal-3BP stimulates or increases interleukin 2 (IL-2) production or expression, and IL-2 is believed to reduce self-reactive T cells. Accordingly, the invention provides, inter alia, Gal-3BP compositions, and methods and uses of Gal-3BP, for treatment of undesirable and aberrant immune responses, immune disorders, inflammatory responses, inflammation and autoimmune responses, disorders and diseases in a subject.

Compositions, methods and uses of the invention include Gal-3BP polypeptide, which is a secreted 585 (murine 577) amino acid protein. Gal-3BP has been reported to be a member of the macrophage scavenger receptor cysteine-rich domain superfamily (Koths et al., J. Biol. Chem. 268:14245 (1993)). Gal-3BP appears to be ubiquitously expressed (Koths et al., J. Biol. Chem. 268:14245 (1993); and Ullrich et al., J. Biol. Chem. 269: 18401 (1994)) and can be detected in many body fluids such as semen, saliva, urine, tears (Koths et al., J. Biol. Chem. 268:14245 (1993)), milk (D'Ostilio et al., Clin. Exp. Immunol. 104: 543 (1996); and Fornarini et al., Clin. Exp. Immunol. 115:91 (1999)) and plasma, where it is associated with microparticles (Smalley et al., Thromb. Haemost. 97:67 (2007)).

A “polypeptide” refers to two, or more, amino acids linked by an amide or equivalent bond. A polypeptide can also be referred to herein, inter alia, as a protein, peptide, or an amino acid sequence. Polypeptides include at least two, or more, amino acids bound by an amide bond, or equivalent. Polypeptides can form intra or intermolecular disulfide bonds. Polypeptides can also form higher order structures, such as multimers or oligomers, with the same or different polypeptide, or other molecules.

A Gal-3BP polypeptide refers to full length polypeptide sequence, as well as subsequences, fragments or portions, and modified forms and variants of Gal-3BP polypeptide, unless the context indicates otherwise. Such Gal-3BP subsequences, fragments, modified forms and variants have at least a part of, a function or activity of an unmodified or reference Gal-3BP protein. In particular embodiments, a modified form or variant retains, at least a part of, a function or activity of an unmodified or reference protein. A “functional polypeptide” or “active polypeptide” refers to a modified polypeptide or a subsequence thereof. For example, a functional or active Gal-3BP polypeptide or a subsequence thereof possesses at least one partial function or activity (e.g., biological activity) characteristic of a native wild type or full length counterpart polypeptide, for example, Gal-3BP, as disclosed herein, which function or activity can be identified through an assay. The invention therefore includes modified forms and variants of Gal-3BP polypeptide sequences, and subsequences, which modified forms or variants typically retain, at least a part of, one or more functions or activities of an unmodified or reference Gal-3BP polypeptide sequence.

As disclosed herein, particular non-limiting examples of a function or activity of Gal-3BP polypeptide is to decrease, reduce, inhibit, suppress, limit or control an undesirable or aberrant immune response, immune disorder, inflammatory response, or inflammation, or an autoimmune response, disorder or disease. Additional non-limiting examples of a function or activity of Gal-3BP polypeptide is to increase, induce or stimulate IL-2 secretion or expression, calcium mobilization, NFAT translocation to nucleus, induction of RNA expression, and decreasing, reducing, inhibiting, suppressing, limiting or controlling proinflammatory cytokine secretion, production or expression (e.g., TNF-α, IL-6, etc.), etc. Such modified Gal-3BP polypeptide sequences, variants and subsequences can have one or more of the foregoing activities, or other functions or activities attributed to wild type native Gal-3BP polypeptide.

As used herein, the term “modify” and grammatical variations thereof, means that the composition deviates from a reference composition. Modifications include, for example, substitutions, additions, insertions, deletions and other variations to the amino acid sequences set forth herein, which can be referred to as “variants.” The invention compositions, methods and uses include such modified Gal-3BP polypeptides.

Exemplary sequence substitutions, additions, and insertions include a full length or a portion of a sequence with one or more amino acids substituted, added or inserted, for example of Gal-3BP polypeptide sequence, wherein the modified or variant Gal-3BP polypeptide decreases, reduces, inhibits, suppresses, limits or controls an undesirable or aberrant immune response, immune disorder, inflammatory response, or inflammation, an autoimmune response, disorder or disease, or increases, induces or stimulates IL-2 secretion or expression, calcium mobilization, NFAT translocation to nucleus, induction or stimulation of RNA expression, decreasing, reducing, inhibiting, suppressing, limiting or controlling proinflammatory cytokine secretion, production or expression (e.g., TNF-α, IL-6, etc.), etc.

Particular non-limiting examples of modified polypeptides include, for example, non-conservative and conservative substitutions of Gal-3BP polypeptide sequences. In particular embodiments, a modified protein has one or a few (e.g., 1-5%, 5-10%, 10-20% or 20-30% of the residues of total protein length, or 2-10, 10-20, 20-30, 30-40, 40-50, 50-60, 60-75, 75-100 residues, substituted) conservative or non-conservative substitutions.

As used herein, the term “conservative substitution” denotes the replacement of an amino acid residue by another, chemically or biologically similar residue. Biologically similar means that the substitution does not destroy a biological activity or function. Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide. Further modified forms include sugars, or glycosylated proteins.

Modified forms of protein (e.g., Gal-3BP polypeptide sequence or subsequence), and other compositions, include additions and insertions. For example, an addition can be one or more amino acid residues, or a covalent or non-covalent attachment of any type of molecule to a protein (e.g., Gal-3BP) or other composition. Particular examples of additions and insertions confer a complementary or a distinct function or activity.

Additions and insertions include fusion polypeptide sequence constructs, which is a sequence (e.g., Gal-3BP) having one or more molecules not normally present in a reference native (wild type) sequence (e.g., Gal-3BP) covalently attached to the sequence. The terms “fusion” or “chimeric” and grammatical variations thereof, when used in reference to a molecule, such as a Gal-3BP, means that a portions or part of the molecule contains a different entity distinct from the molecule (e.g., Gal-3BP) as they do not typically exist together in nature. That is, for example, one portion of the fusion or chimera, such as Gal-3BP, includes or consists of a portion that does not exist together in nature, and is structurally distinct. A particular example is an amino acid sequence of another protein (e.g., immunoglobulin such as an Fc domain, or antibody) attached to produce a chimera, or a chimeric polypeptide, to impart a distinct function (e.g., increased solubility, in vivo half life, etc.).

Additional non-limiting examples of amino acid modifications include protein subsequences and fragments. A subsequence, fragment or portion of Gal-3BP polypeptide means less than the full length reference sequence, which is typically a native full length Gal-3BP polypeptide sequence. Exemplary Gal-3BP subsequences and fragments include a Gal-3BP polypeptide fragment or a portion of that decrease, reduce, inhibit, suppress, limit or control an undesirable or aberrant immune response, immune disorder, inflammatory response, or inflammation, an autoimmune response, disorder or disease, or increases, induces or stimulates IL-2 secretion or expression, calcium mobilization, NFAT translocation to nucleus, induction or stimulation of RNA expression, or decreases, reduces, inhibits, suppresses, limits or controls proinflammatory cytokine secretion, production or expression (e.g., TNF-α, IL-6, etc.), etc.

Non-limiting subsequences of full length Galectin-3 binding protein (Gal-3BP) include amino acids having a length of about 5-10, 10-20, 20-25, 25-50, 50-100, 100-150, 150-200, 200-250, 250-300, 300-350, 350-400, 400-500, 500-600 or more amino acids in length, but less than a full length Gal-3BP polypeptide sequence, e.g., a native (naturally occurring) sequence. Gal-3BP subsequences, fragments and portions can retain all or a part of a function or activity of full length Gal-3BP polypeptide (e.g., decreases, reduces, inhibits, suppresses, limits or controls an undesirable or aberrant immune response, immune disorder, inflammatory response, or inflammation, an autoimmune response, disorder or disease, or increases, induces or stimulates IL-2 secretion or expression, calcium mobilization, NFAT translocation to nucleus, induction or stimulation of RNA expression, or decreasing, reducing, inhibiting, suppressing, limiting or controlling proinflammatory cytokine secretion, production or expression (e.g., TNF-α, IL-6, etc.), etc.

Modified and variant Gal-3BP polypeptide sequences and subsequences of the invention may have an activity or function greater or less than 2-5, 5-10, 10-100, 100-1000 or 1000-10,000-fold activity or function than a comparison Gal-3BP polypeptide sequence or subsequence. For example, a modified Gal-3BP polypeptide sequences or subsequence could have an activity or function greater or less than 2-5, 5-10, 10-100, 100-1000 or 1000-10,000-fold activity or function of a reference Ga13BP to decrease, reduce, inhibit, suppress, limit or control an undesirable or aberrant immune response, immune disorder, inflammatory response, or inflammation, or an autoimmune response, disorder or disease, etc.

Studies set forth herein disclose several Gal-3BP polypeptide subsequences, fragments and portions that retain all or a part of a function or activity of full length Gal-3BP polypeptide. In particular embodiments, Gal-3BP polypeptide subsequences, fragments and portions include residues 24-124 (SRCR domain); residues 153-221 (BTB domain); and residues 260-360 (BACK domain) of Gal-3BP polypeptide. In additional particular embodiments, Gal-3BP polypeptide subsequences, fragments and portions include subsequences of residues 24-124 (SRCR domain), e.g., residues 25-120 of SRCR domain; residues 153-221 (BTB domain), e.g., residues 155-218 of BTB domain; and residues 260-360 (BACK domain), e.g., residues 262-357 of BACK domain.

Gal-3BP polypeptide also refers to polypeptide sequences having sequence identity to a reference Gal-3BP polypeptide sequence. Such Gal-3BP polypeptide sequences can have at least about 60%, 65%, 70%, 75%, 80%, 85%, 90%, 95%, 96%, 97%, 98%, 99% or more identity (homology) to a reference Gal-3BP polypeptide sequence (e.g., a mammalian Gal-3BP polypeptide sequence, such as human Gal-3BP polypeptide sequence). Such Gal-3BP polypeptide sequences with at least about 60%, 65%, 70%, 75%, 80%, 85%, 90%, 95%, 96%, 97%, 98%, 99% or more identity (homology) to a reference Gal-3BP polypeptide sequence can have sufficient identity to retain all or a part of a function or activity of a reference Gal-3BP polypeptide (e.g., decreases, reduces, inhibits, suppresses, limits or controls an undesirable or aberrant immune response, immune disorder, inflammatory response, or inflammation, an autoimmune response, disorder or disease, or increases, induces or stimulates IL-2 secretion or expression, calcium mobilization, NFAT translocation to nucleus, induction or stimulation of RNA expression, or decreasing, reducing, inhibiting, suppressing, limiting or controlling proinflammatory cytokine secretion, production or expression (e.g., TNF-α, IL-6, etc.)).

The term “identity” and grammatical variations thereof, mean that two or more referenced entities are the same. Thus, where two polypeptide sequences (e.g., Gal-3BP polypeptide sequences) are identical, they have the same amino acid sequence, at least within the referenced region or portion. The identity can be over a defined area (region or domain) of the sequence. An “area of identity” refers to a portion of two or more referenced entities that are the same. Thus, where two protein sequences are identical over one or more sequence regions they share identity within that region.

The percent identity can extend over the entire sequence length of the polypeptide (e.g., a Gal-3BP polypeptide sequence). In particular aspects, the length of the sequence sharing the percent identity is 5 or more contiguous amino acids, e.g., 5, 6, 7, 8, 9, 10, 11, 12, 13, 14, 15, 16, 17, 18, 19, 20, 21, 22, 23, 24, 25, etc. contiguous amino acids. In additional particular aspects, the length of the sequence sharing the percent identity is 25 or more contiguous amino acids, e.g., 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet additional particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids.

The extent of identity (homology) between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch-2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

An exemplary full length human Gal-3BP polypeptide sequence (SEQ ID NO:1) is as follows:

```
                10 20 30 40 50 60
MTPPRLFWVW LLVAGTQGVN DGDMRLADGG ATNQGRVEIF YRGQWGTVCD NLWDLTDASV
            70 80 90 100 110 120
VCRALGFENA TQALGRAAFG QGSGPIMLDE VQCTGTEASL ADCKSLGWLK SNCRHERDAG
              130 140 150 160 170 180
VVCTNETRST HTLDLSRELS EALGQIFDSQ RGCDLSISVN VQGEDALGFC GHTVILTANL
              190 200 210 220 230 240
EAQALWKEPG SNVTMSVDAE CVPMVRDLLR YFYSRRIDIT LSSVKCFHKL ASAYGARQLQ
              250 260 270 280 290 300
GYCASLFAIL LPQDPSFQMP LDLYAYAVAT GDALLEKLCL QFLAWNFEAL TQAEAWPSVP
              310 320 330 340 350 360
TDLLQLLLPR SDLAVPSELA LLKAVDTWSW GERASHEEVE GLVEKIRFPM MLPEELFELQ
              370 380 390 400 410 420
FNLSLYWSHE ALFQKKTLQA LEFHTVPFQL LARYKGLNLT EDTYKPRIYT SPTWSAFVTD
              430 440 450 460 470 480
SSWSARKSQL VYQSRRGPLV KYSSDYFQAP SDYRYYPYQS FQTPQHPSFL FQDKRVSWSL
              490 500 510 520 530 540
VYLPTIQSCW NYGFSCSSDE LPVLGLTKSG GSDRTIAYEN KALMLCEGLF VADVTDFEGW
              550 560 570 580
KAAIPSALDT NSSKSTSSFP CPAGHFNGFR TVIRPFYLTN SSGVD
```

Such a full length human Gal-3BP polypeptide sequence, and modified forms and variants as set forth herein, are also included.

Additional representative mammalian (human, Pan troglodytes, Canis lupus familiaris, Bos Taurus, Mus musculus and Rattus norvegicus) sequences (SEQ ID NOs:1-6), and an alignment showing the regions of identity, are illustrated as follows:

| | | | |
|---|---|---|---|
| NP_005558.1 | 1 | -----------------------MTPPRLFWVWLLVA-----------GT | 16 |
| XP_001158328.1 | 1 | -----------------------MTPPRLFWVWLLVA-----------GT | 16 |
| XP_540464.2 | 1 | -----------------------MALPLVLWMCLLVA-----------GT | 16 |
| NP_001039781.1 | 1 | -----------------------MAPLRLFWIWLLVV-----------GT | 16 |
| NP_035280.1 | 1 | -----------------------MALLWLLSVFLLVP-----------GT | 16 |
| NP_620796.1 | 1 | -----------------------MALLWLLSVFLLVP-----------GT | 16 |
| NP_005558.1 | 17 | QGVNDGDMRLADGGATNQGRVEIFYRGQWGTVCDNLWDLTDASVVCRALG | 66 |
| XP_001158328.1 | 17 | QGVNDGDMRLADGGATNQGRVEIFYRGQWGTVCDNLWDLTDASVVCRALG | 66 |
| XP_540464.2 | 17 | QGVKDGDMRLANGDTANEGRVEIFYSGRWGTVCDNLWDLMDASVVCRALG | 66 |
| NP_001039781.1 | 17 | RGVKDGDMRLADGGSANQGRVEIYYNGQWGTVCENMWDLTDASVVCRALG | 66 |
| NP_035280.1 | 17 | QGTEDGDMRLVNGASANEGRVEIFYRGRWGTVCDNLWNLLDAHVVCRALG | 66 |
| NP_620796.1 | 17 | QGAKDGDMRLVNGASASEGRVEIFYRGRWGTVCDNLWNLLDAHVVCRALG | 66 |
| NP_005558.1 | 67 | FENATQALGRAAFGQGSGPIMLDEVQCTGTEASLADCKSLGWLKSNCRHE | 116 |
| XP_001158328.1 | 67 | FENATQALGRAAFGQGSGPIMLDEVQCMGTEASLADCKSLGWLKSNCRHE | 116 |
| XP_540464.2 | 67 | FENATEALGGAAFGPGKGPIMLDEVECTGTEPSLANCTSLGWMKSNCRHN | 116 |
| NP_001039781.1 | 67 | FQNATEALGGAAFGPGYGPIMLDEVRCTGTEPSLANCSSLGWMRSNCRHD | 116 |
| NP_035280.1 | 67 | YENATQALGRAAFGPGKGPIMLDEVECTGTESSLASCRSLGWMVSRCGHE | 116 |
| NP_620796.1 | 67 | YENATQALSRAAFGPGKGPIMLDEVECTGNESSLANCSSLGWMVSHCGHE | 116 |
| NP_005558.1 | 117 | RDAGVVCTNETRSTHTLDLSRE----LSEALGQIFDSQRGCDLSISVN-V | 161 |
| XP_001158328.1 | 117 | RDAGVVCTNETRSTHTLDLSRE----LSEALGQIFDSQRGCDLSISVN-V | 161 |
| XP_540464.2 | 117 | QDAGVVCSNETRGAHTLDLSGE----LPAALEQIFDSQRGCDLSIRVK-V | 161 |
| NP_001039781.1 | 117 | KDASVICTNETRGVYTLDLSGE----LPAALEQIFESQKGCDLFITVK-V | 161 |
| NP_035280.1 | 117 | KDAGVVCSNDTTGLHILDLSGE----LSDALGQIFDSQQGCDLFIQVT-G | 161 |
| NP_620796.1 | 117 | KDAGVVCSNDSRGIHILDLSGE----LPDALGQIFDSQQDCDLFIQVT-G | 161 |
| NP_005558.1 | 162 | QGEDALG--FCGHTVILTANLEAQALWKEPGSNVTMSVDAECVPMVRDLL | 209 |
| XP_001158328.1 | 162 | QGEDALG--FCGHTVILTANLEAQALWKEPGSNVTMSVDAECVPMVRDLL | 209 |
| XP_540464.2 | 162 | KDQEEEGPHFCAHRLILAANPEAQALCKAPGSTVTMEVDAECLPVVRDFI | 211 |
| NP_001039781.1 | 162 | REEDEIA--MCAHKLILSTNPEAHGLWKEPGSRVTMEVDAECVPVVKDFI | 209 |
| NP_035280.1 | 162 | QGYEDLS--LCAHTLILRTNPEAQALWQVVGSSVIMRVDAECMPVVRDFL | 209 |
| NP_620796.1 | 162 | QGHGDLS--LCAHTLILRTNPEAQALWQVVGSSVIMRVDAECMPVVRDFL | 209 |
| NP_005558.1 | 210 | RYFYSRRIDITLSSVKCFHKLASAYGARQLQGYCASLFAILLPQDPSFQM | 259 |
| XP_001158328.1 | 210 | RYFYSRRIDITLSSVKCFHKLASAYGARQLQGYCASLFAILLPRDPSFQT | 259 |
| XP_540464.2 | 212 | RYLYSRRLDISLTSVKCFHKLASAYEAQQLQSFCASLFAILLPEDPSFQA | 261 |
| NP_001039781.1 | 210 | RYLYSRRIDVSLSSVKCLHKFASAYQAKQLQSYCGHLFAILIPQDPSFWT | 259 |
| NP_035280.1 | 210 | RYFYSRRIEVSMSSVKCLHKLASAYGATELQDYCGRLFATLLPQDPTFHT | 259 |
| NP_620796.1 | 210 | RYFYSRRIEVSMSSVKCLHKLASAYGATELQGYCGRLFVTLLPQDPTFHT | 259 |
| NP_005558.1 | 260 | PLDLYAYAVATGDALLEKLCLQFLAWNFEALTQAEAWPSVPTDLLQLLLP | 309 |
| XP_001158328.1 | 260 | PLDLYAYAVATGDALLEKLCLQFLAWNFEALTQAEAWPSVPTDLLQLLLP | 309 |
| XP_540464.2 | 262 | PLDLYAYALATQDPVLEELCVQFLAWNFEGLTQATAWPRVPTALLQLLLS | 311 |
| NP_001039781.1 | 260 | PLELYAYALATRDIVLEEICVQFLAWNFGALTQAEAWPSVPPALLQGLLS | 309 |
| NP_035280.1 | 260 | PLDLYAYARATGDSMLEDLCVQFLAWNFEPLTQSESWSAVPTTLIQALLP | 309 |
| NP_620796.1 | 260 | PLELYEYAQATGDSVLEDLCVQFLAWNFEPLTQAEAWLSVPNALIQALLP | 309 |
| NP_005558.1 | 310 | RSDLAVPSELALLKAVDTWSWGERA--SHEEVEGLVEKIRFPMMLPEELF | 357 |
| XP_001158328.1 | 310 | RSDLAVPSELALLKAVDTWSWGERA--SHEEVEDLVEKIRFPMMLPEELF | 357 |
| XP_540464.2 | 312 | RSELAVPSELALLTALDVWSQERRP--SHGEVARLVDKVRFPMMLPEHLF | 359 |
| NP_001039781.1 | 310 | RTELVVPSELVLLLAVDKWSQERHT--SHKEVEALVGQVRFPMMPPQDLF | 357 |
| NP_035280.1 | 310 | KSELAVSSELDLLKAVDQWSTETIA--SHEDIERLVEQVRFPMMLPQELF | 357 |
| NP_620796.1 | 310 | KSELAVSSELDLLKAVDQWSTATGA--SHGDVERLVEQIRFPMMLPQELF | 357 |
| NP_005558.1 | 358 | ELQFNLS-LYWSHEALFQKKTLQALEFHTVPFQLLARYKGLNLTEDTYKP | 406 |
| XP_001158328.1 | 358 | ELQFNLS-LYWSHEALFQKKTLQALEFHTVPFQLLARYKGLNLTEDTYKP | 406 |
| XP_540464.2 | 360 | ELQFNLS-LYWSHEALFQKKILQALEFHTVPFRLLAQHRGLNLTEDAYQP | 408 |
| NP_001039781.1 | 358 | SLQFNLS-LYWSHEALFQKKILQALEFHTVPFELLAQYWGLNLTEGTYQP | 406 |
| NP_035280.1 | 358 | ELQFNLS-LYQDHQALFQRKTMQALEFHTVPVEVLAKYKGLNLTEDTYKP | 406 |
| NP_620796.1 | 358 | ELQFNLS-LYQGHQALFQRKTMEALEFHTVPLKVLAKYRSLNLTEDVYKP | 406 |
| NP_005558.1 | 407 | RIYTSPTWSAFVTDSSWSARKSQLVYQSRRGPLVKYSSDYFQAPSDYRYY | 456 |
| XP_001158328.1 | 407 | RIYTSPTWSASVTDSSWSARKSQLVYQSRRGPLVKYSSNYFQAPSDYRYY | 456 |
| XP_540464.2 | 409 | RLYTSPTWSASVSRSS----------------------------SRYWNY | 430 |
| NP_001039781.1 | 407 | RLYTSPTWSQSVMSSS--------------------------------YN | 424 |
| NP_035280.1 | 407 | RLYTSSTWSSLVMASTWRAQRYEYNRYNQ--------LYTYGYGSVARYN | 448 |
| NP_620796.1 | 407 | RLYTSSTWSSLLMAGAWSTQSY---KYRQ--------FYTYNYGSQSRYS | 445 |
| NP_005558.1 | 457 | PYQSF-QTPQHPSFLFQDKRVSWSLVYLPTIQSCWNYGFSCSSDELPVLG | 505 |
| XP_001158328.1 | 457 | PYQSF-QTPQHPSFLFQDKRVSWSLVYLPTIQSCWNYGFSCSSDELPVLG | 505 |
| XP_540464.2 | 431 | PYQSF-QTPQHPSFLFQNKYISWSLVYLPTVQSCWNYGFSCSSDEVPLLG | 479 |
| NP_001039781.1 | 425 | PSRSF-QTPQHPSFLFHDSSVSWSFVYLPTLQSCWNYGFSCSSDDPPLLA | 473 |
| NP_035280.1 | 449 | SYQSF-QTPQHPSFLFKDKQISWSATYLPTMQSCWNYGFSCTSNELPVLG | 497 |
| NP_620796.1 | 446 | SYQNF-QTPQHPSFLFKDKLISWSATYLPTIQSCWNYGFSCTSDELPVLG | 494 |
| NP_005558.1 | 506 | LTKSGG--SDRTIAYENKALMLCEGL-FVADVTDFEGWKAAIPSALDTNS | 552 |
| XP_001158328.1 | 506 | LTKSGG--SDRTIAYENKALMLCEGL-FVADVTDFEGWKAAIPSALDINS | 552 |
| XP_540464.2 | 480 | LSKSDY--SDPTIGYENKALMRCGGR-FVADVTDFEGQKALIPSALGTNS | 526 |
| NP_001039781.1 | 474 | LSKSSYSKSNPTIGYENRALLHCEGS-FVVDVIDFKGWKALVPSALATNS | 522 |
| NP_035280.1 | 498 | LTTSSY--SNPTIGYENRVLILCGGY-SVVDVTSFEGSKAPIPTALDTNS | 544 |
| NP_620796.1 | 495 | LTTSSY--SDPTIGYENKALILCGGY-SVVDVTTFIGSKAPIPGTQETNS | 541 |
| NP_005558.1 | 553 | SKSTSSFPCPAGHFNGFRTVIRPFYLTNSSGVD | 585 |
| XP_001158328.1 | 553 | SKSTSSFPCPAGHFNGFRTVIRPFYLTNSSGVD | 585 |
| XP_540464.2 | 527 | SRRPSLFPCLGGSFSSFQVVIRPFYLTNSSDVD | 559 |
| NP_001039781.1 | 523 | SRSTSLFPCPSGVFSRFQVVIRPFYLTNSTDMD | 555 |
| NP_035280.1 | 545 | SKTPSLFPCASGAFSSFRVVIRPFYLTNSTDMV | 577 |
| NP_620796.1 | 542 | SKTPSLFPCASGAFSSFREVIRPFYLTNSTDTE | 574 |

| | | | % Identity vs. |
|---|---|---|---|
| | | | Homo sapiens |
| Protein Acc. | Gene | Organism | (protein) |
| NP_005558.1 | LGALS3BP | Homo sapiens | |
| XP_001158328.1 | LGALS3BP | Pan troglodytes | 98.8 |
| XP_540464.2 | LGALS3BP | Canis lupus familiaris | 76.1 |
| NP_001039781.1 | LGALS3BP | Bos taurus | 72.0 |
| NP_035280.1 | Lgals3bp | Mus musculus | 69.8 |
| NP_620796.1 | Lgals3bp | Rattus norvegicus | 68.1 |

Such full length mammalian Gal-3BP polypeptide sequences, and modified forms and variants as set forth herein, are also included as invention compositions, methods and uses.

Modifications can be produced using methods known in the art (e.g., PCR based site-directed, deletion and insertion mutagenesis, chemical modification and mutagenesis, cross-linking, etc.), or may be spontaneous or naturally occurring (e.g. random mutagenesis). For example, naturally occurring Gal-3BP polypeptide sequence allelic variants can occur by alternative RNA splicing, polymorphisms, or spontaneous mutations of a nucleic acid encoding Gal-3BP polypeptide. Further, deletion of one or more amino acids can also result in a modification of the structure of the resultant polypeptide without significantly altering a biological function or activity. Deletion of amino acids can lead to a smaller active molecule. For example, as set forth herein, removal of certain Gal-3BP polypeptide amino acids does not destroy the ability to decrease, reduce, inhibit, suppress, limit or control an undesirable or aberrant immune response, immune disorder, inflammatory response, or inflammation, an autoimmune response, disorder or disease, or to increase, induce or stimulate IL-2 secretion or expression, calcium mobilization, NFAT translocation to nucleus, induction or stimulation of RNA expression, or to decrease, reduce, inhibit, suppress, limit or control proinflammatory cytokine secretion, production or expression (e.g., TNF-α, IL-6, etc.), etc.

Invention compositions, methods and uses include isolated and purified Gal-3BP polypeptides, as well as modified forms and variants, such as subsequences and fragments of Gal-3BP polypeptides. The term “isolated,” when used as a modifier of a composition (e.g., Gal-3BP polypeptide sequences, subsequences, modified forms, variants, etc.), means that the compositions are made by the hand of man or are separated, completely or at least in part, from their naturally occurring in vivo environment. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man.

An “isolated” composition (e.g., a Gal-3BP polypeptide sequence) can also be “substantially pure” or “purified” when free of most or all of the materials with which it typically associates with in nature. Generally, isolated compositions are substantially free of one or more materials with which they normally associate with in nature, for example, one or more protein, nucleic acid, lipid, carbohydrate, cell membrane. Thus, an isolated sequence that also is substantially pure or purified does not include polypeptides or polynucleotides present among millions of other sequences, such as antibodies of an antibody library or nucleic acids in a genomic or cDNA library, for example. Typically, purity can be at least about 50%, 60% or more by mass. The purity can also be about 70% or 80% or more, and can be greater, for example, 90% or more. Purity can be determined by any appropriate method, including, for example, UV spectroscopy, chromatography (e.g., HPLC, gas phase), gel electrophoresis and sequence analysis (nucleic acid and peptide), and is typically relative to the amount of impurities, which typically does not include inert substances, such as water.

A “substantially pure” or “purified” composition can be combined with one or more other molecules. Thus, “substantially pure” or “purified” does not exclude combinations of compositions, such as combinations of Gal-3BP polypeptide sequences, subsequences, antibodies, agents, drugs or therapies. For example, a composition can include a combination of Gal-3BP and an anti-inflammatory drug or agent.

As used herein, the term “recombinant,” when used as a modifier of polypeptides, means that the compositions have been manipulated (i.e., engineered) in a fashion that generally does not occur in nature (e.g., in vitro). A particular example of a recombinant polypeptide would be where a Gal-3BP polypeptide is expressed by a cell transfected with a polynucleotide encoding the Gal-3BP polypeptide. Another example of a recombinant polypeptide is a hybrid or fusion sequence, such as a chimeric Gal-3BP polypeptide sequence comprising and a second sequence, such as a heterologous functional domain.

Invention compositions, methods and uses that include Gal-3BP polypeptide can include any amount or dose of Gal-3BP polypeptide, subsequence, modified form or variant. In particular embodiments, Gal-3BP is in a concentration range of about 10 μg/ml to 100 mg/ml, or in a range of about 100 μg/ml to 1,000 mg/ml, or at a concentration of about 1 mg/ml. In further particular embodiments, Gal-3BP is in an amount of 10-1,000 milligrams, or an amount of 10-100 milligrams.

Gal-3BP polypeptides, subsequences and modified forms and variants as disclosed herein (e.g., modified or unmodified full length native mammalian, such as human Gal-3BP polypeptide sequences), including compositions including Gal-3BP, are useful in various treatment methods and uses. Such Gal-3BP and compositions thereof are applicable to treating and uses for numerous disorders and diseases, both chronic and acute.

Responses, disorders and diseases treatable in accordance with the invention include, but are not limited to, treatment of acute and chronic undesirable or aberrant immune response, immune disorder, inflammatory response, or inflammation. Responses, disorders and diseases treatable in accordance with the invention also include, but are not limited to treatment of an acute and chronic autoimmune response, disorder or disease. Such responses, disorders and diseases may be antibody or cell mediated, or a combination of antibody and cell mediated.

In accordance with the invention, there are provided compositions, methods and uses for treating acute and chronic responses, disorders and diseases in which a subject would benefit from a Gal-3BP polypeptide sequence. In one embodiment, a method includes administering a Gal-3BP polypeptide sequence to a subject, having or at risk of acute or chronic immune response, immune disorder, inflammatory response, or inflammation, an autoimmune response, disorder or disease, in an amount sufficient to treat the acute or chronic undesirable or aberrant immune response, immune disorder, inflammatory response, or inflammation, an autoimmune response, disorder or disease.

In particular aspects, a method decreases, reduces, inhibits, suppresses, limits or controls an undesirable or aberrant immune response, immune disorder, inflammatory response, or inflammation in a subject. In additional particular aspects, a method decreases, reduces, inhibits, suppresses, limits or controls an autoimmune response, disorder or disease in a subject. In further particular aspects, a method decreases, reduces, inhibits, suppresses, limits or controls an adverse symptom of the undesirable or aberrant immune response, immune disorder, inflammatory response, or inflammation, or an adverse symptom of the autoimmune response, disorder or disease.

As used herein, an “undesirable or aberrant” immune response, inflammatory response, or inflammation, and grammatical variations thereof, can be a normal response, function or activity. Thus, normal immune responses, inflammatory responses, and inflammation that are not considered aberrant so long as they are undesirable are included within the meaning of these terms. An undesirable immune response, inflammatory response, or inflammation can also be an aberrant or abnormal response. An aberrant or abnormal immune response, inflammatory response, or inflammation deviates from normal. An aberrant or abnormal immune response, inflammatory response, or inflammation or disorder can be humoral or cellular in nature, or both, either chronic or acute.

Undesirable or aberrant immune responses, inflammatory responses, or inflammation are characterized by many different physiological adverse symptoms or complications, which can be a result be humoral, cell-mediated or a combination thereof. For example, an undesirable or aberrant immune response, inflammatory response, or inflammation can result in destruction of cells, tissue or organ, such as Crohn's disease, inflammatory bowel disease (IBD) or ulcerative colitis, or a transplant, as in graft vs. host disease. Responses, disorders and diseases that can be treated in accordance with the invention include, but are not limited to, those that cause cell or tissue/organ damage in a subject.

At the whole body, regional or local level, an immune response, inflammatory response, or inflammation can be characterized by swelling, pain, headache, fever, nausea, skeletal joint stiffness or lack of mobility, rash, redness or other discoloration. At the cellular level, an immune response, inflammatory response, or inflammation can be characterized by one or more of cell infiltration of the region, production of antibodies (e.g., auto-antibodies), production of cytokines, lymphokines, chemokines, interferons and interleukins, cell growth and maturation factors (e.g., proliferation and differentiation factors), cell accumulation or migration and cell, tissue or organ damage. Thus, methods and uses of the invention include treatment of and an ameliorative effect upon any such physiological symptoms or cellular responses characteristic of immune responses, inflammatory response, or inflammation.

Autoimmune responses, disorders and diseases are generally characterized as an undesirable or aberrant response, activity or function of the immune system characterized by increased or undesirable humoral or cell-mediated immune responsiveness or memory, or decreased or insufficient tolerance to self-antigens. Autoimmune responses, disorders and diseases that may be treated in accordance with the invention include but are not limited to responses, disorders and diseases that cause cell or tissue/organ damage in the subject.

Examples of adverse symptoms of an undesirable or aberrant immune response, immune disorder, inflammatory response, or inflammation, or an adverse symptom of the autoimmune response, disorder or disease include swelling, pain, rash, headache, fever, nausea, diarrhea, bloat, lethargy, skeletal joint stiffness, or tissue or cell damage. Additional examples of adverse symptoms of an autoimmune response, disorder or disease include a decrease or reduction of auto-antibodies, of pro-inflammatory cytokines or chemokines (e.g., TNF-alpha, IL-6, etc.) improved tolerance for auto-antigens, etc. Examples of adverse symptoms occur in particular tissues, or organs, or regions or areas of the body, such as in an epidermal or mucosal tissue, gut, bowel, pancreas, thymus, liver, kidney, muscle, central or peripheral nerves, spleen, skin, or a skeletal joint (e.g., knee, ankle, hip, shoulder, wrist, finger, toe, or elbow).

Specific non-limiting examples of aberrant or undesirable immune disorders, inflammatory responses, inflammation, autoimmune responses, disorders and diseases, treatable in accordance with the invention include: rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (γ_{c}) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), and hyper IgE syndrome.

Methods and uses include stimulating, promoting, increasing or inducing interleukin-2 (IL-2) production by or secretion from macrophages or dendritic cells. Methods and uses also include stimulating, promoting, increasing or inducing survival, proliferation or differentiation of regulatory T cells (T regs).

Methods and uses further include stimulating, promoting, increasing or inducing nuclear factor of activated T cells (NFAT, NFATC1 and/or NFATC2) activation or translocation into nucleus of a cell. NFAT activation can be indicated, for example, by stimulated, promoted, increased or induced macrophage microRNA expression, such as has-miR-21, has-miR-23b, has-miR-24, has-miR-27a or has-miR-125a-5p.

Methods and uses moreover include decreasing, reducing or inhibiting production, expression or secretion of a pro-inflammatory cytokine (e.g., TNF-α or interleukin-6, IL-6) or interleukin-10 (IL-10), for example, by splenocytes or macrophages.

In various non-limiting aspects of such methods and uses, a Galectin-3 binding protein (BTBD17B) polypeptide or fragment thereof is administered to a subject in an amount intended to achieve one or more of: stimulating, promoting, increasing or inducing interleukin-2 (IL-2) production by or secretion from macrophages or dendritic cells; stimulating, promoting, increasing or inducing survival, proliferation or differentiation of regulatory T cells (T regs); increasing or inducing nuclear factor of activated T cells (NFAT) activation or translocation into nucleus of a cell; stimulating, promoting, increasing or inducing macrophage microRNA expression, such as has-miR-21, has-miR-23b, has-miR-24, has-miR-27a or has-miR-125a-5p; or decreasing, reducing or inhibiting TNF-alpha or IL-10 or IL-6 production or secretion by splenocytes or macrophages.

In various embodiments of the invention, a method results in increasing the amount Gal-3BP polypeptide sequence in the subject, thereby effecting treatment of the subject. Amounts may vary depending upon the subject, the desired effect, and the disorder or disease, or risk of disorder or disease, to be treated. Amounts of Gal-3BP polypeptide can be reflected in blood or plasma. In particular aspects, Gal-3BP polypeptide sequence increases to an amount in the subject of greater than 2 ug/ml in blood plasma, increases to an amount in the subject of greater than 5 ug/ml in blood plasma, increases to an amount in the subject of greater than 10 ug/ml in blood plasma, increases to an amount in the subject of greater than 15 ug/ml in blood plasma, or increases to an amount in the subject of greater than 20 ug/ml in blood plasma. Increased amounts of Gal-3BP polypeptide sequence may be transient, or longer term (e.g., minutes, hours, days, weeks, etc.). In particular aspects, Gal-3BP polypeptide sequence increases to an amount for a period of time greater than 12, 24, 36, 48, 72 hours, or at least 3, 4, 5, 6, 7, 8, 9, 10, 12, 14 days, weeks or months.

The term “contacting” means direct or indirect binding or interaction between two or more entities (e.g., between a Gal-3BP polypeptide sequence and a target). A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration, or delivery.

In methods and uses of the invention, Gal-3BP or a Gal-3BP composition can be administered prior to, substantially contemporaneously with or following an undesirable or aberrant immune response, immune disorder, inflammatory response, or inflammation, or an autoimmune response, disorder or disease, or one or more adverse symptoms, disorders, illnesses, pathologies, diseases, or complications caused by or associated with the foregoing. Thus, methods and uses of the invention may be practiced prior to (i.e. prophylaxis), concurrently with or after evidence of the response, disorder or disease begins (e.g., one or more symptoms of an undesirable or aberrant immune response, immune disorder, inflammatory response, inflammation, or an autoimmune response, disorder or disease), or one or more adverse symptoms, disorders, illnesses, pathologies, diseases, or complications caused by or associated with the undesirable or aberrant immune response, immune disorder, inflammatory response, inflammation or an autoimmune response, disorder or disease. Administering Gal-3BP or a composition thereof prior to, concurrently with or immediately following development of an adverse symptom may decrease, reduce, inhibit, suppress, limit or control the occurrence, frequency, severity, progression, or duration of one or more adverse symptoms, disorders, illnesses, pathologies, diseases, or complications caused by or associated with the undesirable or aberrant immune response, immune disorder, inflammatory response, inflammation or autoimmune response, disorder or disease.

In addition, administering Gal3-BP or a composition thereof prior to, concurrently with or immediately following development of one or more adverse symptoms may decrease, reduce, inhibit, suppress, limit, control or prevent damage to cells, tissues or organs that occurs, for example, due to one or more adverse symptoms, disorders, illnesses, pathologies, diseases, or complications caused by or associated with the undesirable or aberrant immune response, immune disorder, inflammatory response, inflammation or autoimmune response, disorder or disease.

The invention provides combinations of Gal-3BP and a second agent or drug. In one embodiment, a composition includes Gal-3B and an anti-inflammatory agent or drug.

According to the invention, Gal3-BP or a composition thereof can be administered in combination with a second agent, drug or treatment, such as an immunosuppressive, anti-inflammatory, or palliative agent, drug or treatment. According to the invention, Gal3-BP or a composition thereof can be administered prior to, substantially contemporaneously with or following administering a second agent, drug or treatment, such as an immunosuppressive, anti-inflammatory, or palliative agent, drug or treatment.

Non-limiting examples of second agents and drugs include anti-inflammatory agents, such as steroidal and non-steroidal anti-inflammatory drugs (NSAIDs) to limit or control inflammatory symptoms. Exemplary NSAIDs include, without limitation, ibuprofen (2-(isobutylphenyl)-propionic acid); methotrexate (N-[4-(2,4 diamino 6-pteridinyl-methyl]methylamino]benzoyl)-L-glutamic acid); aspirin (acetylsalicylic acid); salicylic acid; diphenhydramine (2-(diphenylmethoxy)-N,N-dimethylethylamine hydrochloride); naproxen (2-naphthaleneacetic acid, 6-methoxy-9-methyl-, sodium salt, (−)); ketorolac (1H-Pyrrolizine-1-carboxylic acid, 2,3-dihydro-5-benzoyl-, (+−)); phenylbutazone (4-butyl-1,2-diphenyl-3,5-pyrazolidinedione); sulindac-(2)-5-fluoro-2-methyl-1-[[p-(methylsulfinyl)phenyl]methylene[-1-H-indene-3-acetic acid; diflunisal (2′,4′,-difluoro-4-hydroxy-3-biphenylcarboxylic acid; piroxicam (4-hydroxy-2-methyl-N-2-pyridinyl-2H-1,2-benzothiazine-2-carboxamide 1,1-dioxide, an oxicam; indomethacin (1-(4-chlorobenzoyl)-5-methoxy-2-methyl-H-indole-3-acetic acid); meclofenamate sodium (N-(2,6-dichloro-m-tolyl)anthranilic acid, sodium salt, monohydrate); ketoprofen (2-(3-benzoylphenyl)-propionic acid; tolmetin sodium (sodium 1-methyl-5-(4-methylbenzoyl-1H-pyrrole-2-acetate dihydrate); diclofenac sodium (2-[(2,6-dichlorophenyl)amino]benzeneatic acid, monosodium salt); hydroxychloroquine sulphate (2-{[4-[(7-chloro-4-quinolyl)amino]pentyl]ethylamino}ethanol sulfate (1:1); penicillamine (3-mercapto-D-valine); flurbiprofen ([1,1-b]phenyl]-4-acetic acid, 2-fluoro-alphamethyl-, (+−)); cetodolac (1-8-diethyl-13,4,9, tetrahydropyrano-[3-4-13]indole-1-acetic acid; mefenamic acid (N-(2,3-xylyl)anthranilic acid; and diphenhydramine hydrochloride (2-diphenyl methoxy-N,N-di-methylethamine hydrochloride).

Immunosuppressive corticosteroids include (steroid receptor agonists) such as budesonide, prednisone, flunisolide. Anti-inflammatory agents also include flunisolide hydrofluoroalkane, estrogen, progesterone, dexamethasone and loteprednol; beta-agonists (e.g., short or long-acting) such as bambuterol, formoterol, salmeterol, albuterol; anticholinergics such as ipratropium bromide, oxitropium bromide, cromolyn and calcium-channel blocking agents; antihistamines such as terfenadine, astemizole, hydroxyzine, chlorpheniramine, tripelennamine, cetirizine, desloratadine, mizolastine, fexofenadine, olopatadine hydrochloride, norastemizole, levocetirizine, levocabastine, azelastine, ebastine and loratadine; antileukotrienes (e.g., anti-cysteinyl leukotrienes (CysLTs)) such as oxatomide, montelukast, zafirlukast and zileuton; phosphodiesterase inhibitors (e.g., PDE4 subtype) such as ibudilast, cilomilast, BAY 19-8004, theophylline (e.g., sustained-release) and other xanthine derivatives (e.g., doxofylline); thromboxane antagonists such as seratrodast, ozagrel hydrochloride and ramatroban; prostaglandin antagonists such as COX-1 and COX-2 inhibitors (e.g., celecoxib and rofecoxib), aspirin; and potassium channel openers.

Additional non-limiting examples of classes of other agents and drugs include anti-inflammatory agents that are immunomodulatory therapies, such as pro-inflammatory cytokine antagonists, such as TNFα antagonists (e.g. etanercept) and the anti-IL-6 receptor tocilizumab; immune cell antagonists, such as the B cell depleting agent rituximab and the T cell costimulation blocker abatacept, which have been used to treat rheumatoid arthritis, and antibodies that bind to cytokines, such as anti-IgE (e.g., rhuMAb-E25 omalizumab), and anti-TNFα, IFNγ, IL-1, IL-2, IL-5, IL-6, IL-9, IL-13, IL-16, and growth factors such as granulocyte/macrophage colony-stimulating factor.

Accordingly, the invention provides Gal-3BP combinations with such agents, drugs and treatments, and methods and uses of such combinations. The invention also provides methods and uses of such Gal-3BP, which are administered prior to, substantially contemporaneously with or following administering a second agent, drug or treatment, such as an immunosuppressive, anti-inflammatory, or palliative agent, drug or treatment.

Invention compositions, methods and uses, such as treatment methods and uses, can provide a detectable or measurable therapeutic benefit or improvement to a subject. A therapeutic benefit or improvement is any measurable or detectable, objective or subjective, transient, temporary, or longer-term benefit to the subject or improvement in the response, disorder or disease, or one or more adverse symptoms, disorders, illnesses, pathologies, diseases, or complications caused by or associated with the undesirable or aberrant response, disorder or disease. Therapeutic benefits and improvements include, but are not limited to, decreasing, reducing, inhibiting, suppressing, limiting or controlling the occurrence, frequency, severity, progression, or duration of an adverse symptom, such as swelling, pain, rash, headache, fever, nausea, diarrhea, bloat, lethargy, skeletal joint stiffness, or tissue or cell damage. Therapeutic benefits and improvements also include, but are not limited to, decreasing, reducing, inhibiting, suppressing, limiting or controlling amounts or activity of auto-antibodies, pro-inflammatory cytokines or chemokines. Therapeutic benefits and improvements further include, but are not limited to increasing or improving tolerance towards self-antigens (i.e., self- or auto-antigens). Compositions, methods and uses of the invention therefore include providing a therapeutic benefit or improvement to a subject.

Compositions, methods and uses of the invention, in which a therapeutic benefit or improvement is a desired outcome, a composition of the invention such as a Gal-3BP polypeptide sequence, can be administered in a sufficient or effective amount to a subject in need thereof. An “effective amount” or “sufficient amount” refers to an amount that provides, in single or multiple doses, alone or in combination, with one or more other compositions (therapeutic agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

For example, a sufficient amount of a Gal-3BP polypeptide or composition thereof, is considered as having a therapeutic effect if administration results in a decreased, reduced, inhibited, or limit of the amount or frequency of therapy for treatment of an undesirable or aberrant immune response, immune disorder, inflammatory response, inflammation or autoimmune response, disorder or disease, or one or more adverse symptoms, disorders, illnesses, pathologies, diseases, or complications caused by or associated with the undesirable or aberrant immune response, immune disorder, inflammatory response, inflammation or autoimmune response, disorder or disease.

The doses of an “effective amount” or “sufficient amount” for treatment (e.g., to provide a therapeutic benefit or improvement) typically are effective to ameliorate a response, disorder or disease, or one, multiple or all adverse symptoms, consequences or complications of the response, disorder or disease, one or more adverse symptoms, disorders, illnesses, pathologies, diseases, or complications, for example, caused by or associated with an undesirable or aberrant immune response, immune disorder, inflammatory response, inflammation or autoimmune response, disorder or disease, to a measurable extent, although decreasing, reducing, inhibiting, suppressing, limiting or controlling progression or worsening of the response, disorder or disease, or an adverse symptom thereof, is a satisfactory outcome.

The term “ameliorate” means a detectable or measurable improvement in a subject's condition or an underlying cellular response. A detectable or measurable improvement includes a subjective or objective decrease, reduction, inhibition, suppression, limit or control in the occurrence, frequency, severity, progression, or duration of the response, disorder or disease, such as an undesirable or aberrant immune response, immune disorder, inflammatory response, inflammation or autoimmune response, disorder or disease, or one or more adverse symptoms, disorders, illnesses, pathologies, diseases, or complications caused by or associated with an undesirable or aberrant immune response, immune disorder, inflammatory response, inflammation or autoimmune response, disorder or disease, or an improvement in an underlying cause or a consequence of the response, disorder or disease e.g., an undesirable or aberrant immune response, immune disorder, inflammatory response, inflammation or autoimmune response, disorder or disease, or a reversal of the response, disorder or disease such as undesirable or aberrant immune response, immune disorder, inflammatory response, inflammation or autoimmune response, disorder or disease. Such improvements can also occur at the cellular level.

Treatment can therefore result in decreasing, reducing, inhibiting, suppressing, limiting, controlling or preventing a response, disorder or disease, or an associated adverse symptom or consequence, or underlying cause; decreasing, reducing, inhibiting, suppressing, limiting, controlling or preventing a progression or worsening of a response, disorder or disease, symptom or consequence, or underlying cause; or further deterioration or occurrence of one or more additional adverse symptoms of the response, disorder or disease. Thus, a successful treatment outcome can lead to a “therapeutic effect,” or “benefit” of decreasing, reducing, inhibiting, suppressing, limiting, controlling or preventing the occurrence, frequency, severity, progression, or duration of an undesirable or aberrant immune response, immune disorder, inflammatory response, inflammation or autoimmune response, disorder or disease, or one or more adverse symptoms or underlying causes or consequences of the undesirable or aberrant immune response, immune disorder, inflammatory response, inflammation or autoimmune response, disorder or disease in the subject. Treatment methods affecting one or more underlying causes of the response, disorder or disease or adverse symptom are therefore considered to be beneficial. Stabilizing a response, disorder or disease, or an adverse symptom thereof, is also a successful treatment outcome.

A therapeutic benefit or improvement therefore need not be complete ablation of the response, disorder or disease, or any one, most or all adverse symptoms, complications, consequences or underlying causes associated with the response, disorder or disease. Thus, a satisfactory endpoint is achieved when there is an incremental improvement in a subject's response, disorder or disease, or a partial decrease, reduction, inhibition, suppression, limit, control or prevention in the occurrence, frequency, severity, progression, or duration, or inhibition or reversal, of the response, disorder or disease, or one or more adverse symptoms or complications or consequences or underlying causes thereof, worsening or progression of the response, disorder or disease (e.g., stabilizing one or more symptoms or complications of the response, disorder or disease), such as undesirable or aberrant immune response, immune disorder, inflammatory response, inflammation or autoimmune response, disorder or disease, or one or more adverse symptoms, disorders, illnesses, pathologies, diseases, or complications caused by or associated with undesirable or aberrant immune response, immune disorder, inflammatory response, inflammation or autoimmune response, disorder or disease, over a short or long duration of time (hours, days, weeks, months, etc.).

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the response, disorder, or disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered sufficient also include amounts that result in a reduction of the use of another treatment, therapeutic regimen or protocol.

An effective amount or a sufficient amount need not be effective in each and every subject treated, prophylactically or therapeutically, nor a majority of treated subjects in a given group or population. An effective amount or a sufficient amount means effectiveness or sufficiency in a particular subject, not a group or the general population. As is typical for such methods, some subjects will exhibit a greater response, or less or no response to a treatment method.

Particular non-limiting examples of therapeutic benefit or improvement for a response, disorder or disease include, for example, decreasing, reducing, inhibiting, suppressing, limiting, controlling, or preventing occurrence, frequency, severity, progression, or duration, or stabilizing or preventing a worsening or progression the response, disorder or disease of the undesirable or aberrant immune response, immune disorder, inflammatory response, inflammation or autoimmune response, disorder or disease, or one or more adverse symptoms thereof. Adverse symptoms caused by or associated with undesirable or aberrant immune response, immune disorder, inflammatory response, inflammation or autoimmune response, disorder or disease, are disclosed herein and are known to one of skill in the art.

A therapeutic benefit can also include reducing susceptibility of a subject to an undesirable or aberrant immune response, immune disorder, inflammatory response, inflammation or autoimmune response, disorder or disease, or accelerate recovery from one or more adverse symptoms, disorders, illnesses, pathologies, diseases, or complications caused by or associated with an undesirable or aberrant immune response, immune disorder, inflammatory response, inflammation or autoimmune response, disorder or disease.

Effectiveness of a treatment method, such as a therapeutic benefit or improvement for a disorder or disease, can be ascertained by various methods. Such methods include, for example, scores measuring swelling, pain, rash, headache, fever, nausea, diarrhea, bloat, lethargy, skeletal joint stiffness, or tissue or cell damage. Measuring antibodies, cytokines or chemokines using various immunological assays, such as ELISA. Determining the degree of tissue or cell damage can be ascertained by CT scanning, MRI, ultrasound, molecular contrast imaging, or molecular ultrasound contrast imaging. For gastrointestinal tract, inflammation can be assessed by endoscopy (colonoscopy, gastroscopy, ERCP), for example. For inflammation of the central nervous system (CNS), cells and cytokines in spinal tap reflect inflammation, for example. CNS inflammation (MS, Parkinson's, Alzheimer's) may be reflected in the corresponding clinical function scores known in the art, for example. Peripheral nerve inflammation can include functional assessment (motor and sensor), for example.

As is typical for treatment or therapeutic methods, some subjects will exhibit greater or less response to a given treatment, therapeutic regiment or protocol. Thus, appropriate amounts will depend upon the response, disorder or disease treated (e.g., the type, status, extent or severity of undesirable or aberrant immune response, immune disorder, inflammatory response, inflammation or autoimmune response, disorder or disease), the therapeutic effect desired, as well as the individual subject (e.g., overall health, the bioavailability, gender, age, etc.).

The term “subject” refers to animals, typically mammalian animals, such as humans, non human primates (apes, gibbons, chimpanzees, orangutans, macaques), domestic animals (dogs and cats), farm animals (horses, cows, goats, sheep, pigs) and experimental animal (mouse, rat, rabbit, guinea pig). Subjects include animal disease models, for example, animal models of undesirable or aberrant immune response, immune disorder, inflammatory response, inflammation or autoimmune response, disorder or disease, etc. or for studying in vivo a Gal-3BP or composition thereof in a method of the invention.

Subjects appropriate for treatment include those that have (existing), have had, or are at risk of having an undesirable or aberrant immune response, immune disorder, inflammatory response, inflammation or autoimmune response, disorder or disease. Such subjects include those undergoing treatment for an undesirable or aberrant immune response, immune disorder, inflammatory response, inflammation or autoimmune response, disorder or disease, as well as those who have had or have undergone treatment or therapy for an undesirable or aberrant immune response, immune disorder, inflammatory response, inflammation or autoimmune response, disorder or disease. Specific non-limiting examples include subjects that have had or are at risk for an undesirable or aberrant immune response, immune disorder, inflammatory response, inflammation or autoimmune response, disorder or disease.

Candidate subjects include those that have or are at increased risk of an undesirable or aberrant immune response, immune disorder, inflammatory response, or inflammation, or an autoimmune response, disorder or disease. A candidate subject, for example, has an undesirable or aberrant immune response, immune disorder, inflammatory response, or inflammation, or an autoimmune response, disorder or disease, or is being treated with a therapy or drug for treatment of an undesirable or aberrant immune response, immune disorder, inflammatory response, or inflammation, or an autoimmune response, disorder or disease. Candidate subjects also include subjects in need of treatment for an undesirable or aberrant immune response, immune disorder, inflammatory response, or inflammation, or an autoimmune response, disorder or disease.

“At risk” subjects typically have increased risk factors for an undesirable or aberrant immune response, immune disorder, inflammatory response, inflammation or autoimmune response, disorder or disease. Particular subjects at risk include subjects that have in the past had an undesirable or aberrant immune response, immune disorder, inflammatory response, or inflammation, or an autoimmune response, disorder or disease. Particular subjects at risk also include subjects prescribed or candidates for treatment or therapy of an undesirable or aberrant immune response, immune disorder, inflammatory response, inflammation or autoimmune response, disorder or disease.

Subjects that optionally can be excluded from methods or uses include a subject diagnosed for an adverse cardiovascular event or cardiovascular disease (for example, coronary artery disease, peripheral artery disease, cerebrovascular disease, or renal artery disease). Additional subjects that optionally can be excluded from methods or uses include those that have had a stroke, myocardial infarction (heart attack), ischemic heart failure, transient ischemic attack or brain trauma. Further subjects that optionally can be excluded from methods or uses include those that have had a treatment or therapy for artherosclerotic plaque formation, elevated blood cholesterol or a cardiovascular disease, or that have been diagnosed for cancer, treated for cancer, or is are remission from cancer.

Gal-3BP and compositions thereof may be contacted or provided in vitro, ex vivo or administered or delivered in vivo. Gal-3BP and compositions thereof can be administered or delivered to provide the intended effect, as single or multiple dosages, for example, in an effective or sufficient amount. Exemplary doses range from about 25-250, 250-500, 500-1000, 1000-2500 or 2500-5000, 5000-25,000, 5000-50,000 pg/kg; from about 50-500, 500-5000, 5000-25,000 or 25,000-50,000 ng/kg; and from about 25-250, 250-500, 500-1000, 1000-2500 or 2500-5000, 5000-25,000, 5000-50,000 mg/kg, on consecutive days, alternating days or intermittently.

Single or multiple doses can be administered on the same or consecutive days, alternating days or intermittently. For example, a Gal-3BP polypeptide can be administered one, two, three, four or more times daily, on alternating days, bi-weekly, weekly, monthly, bi-monthly, or annually. Gal-3BP and compositions thereof can be administered for any appropriate duration, for example, for period of 1 hour, or less, e.g., 30 minutes or less, 15 minutes or less, 5 minutes or less, or 1 minute or less.

Gal-3BP and compositions thereof can be administered to a subject and methods may be practiced substantially contemporaneously with, or within about 1-60 minutes, hours (e.g., within 1, 2, 3, 4 or 5 hours), or days of the onset of an undesirable or aberrant immune response, immune disorder, inflammatory response, inflammation or autoimmune response, disorder or disease.

Compositions and compounds such as a Gal-3BP polypeptide sequence can be administered and methods and uses may be practiced via systemic, regional or local administration, by any route. For example, a Gal-3BP polypeptide may be administered systemically, regionally or locally (e.g., into a region or site of inflammation) via injection, via infusion, by catheter, enema, intravenously, intraarterially, orally (e.g., ingestion or intranasal or inhalation), intramuscularly, intraperitoneally, intradermally, subcutaneously, intracavity, intrarectally, intracranially, topically, transdermally, optically, parenterally, e.g. transmucosally. Methods and uses of the invention including pharmaceutical formulations can be administered via a (micro)encapsulated delivery system or packaged into an implant for administration.

Compositions and compounds such as a Gal-3BP polypeptide sequence, including combinations of Gal-3BP and other drugs or agents, and methods include pharmaceutical compositions, which refer to “pharmaceutically acceptable” and “physiologically acceptable” carriers, diluents or excipients. As used herein, the term “pharmaceutically acceptable” and “physiologically acceptable,” when referring to carriers, diluents or excipients includes solvents (aqueous or non-aqueous), detergents, solutions, emulsions, dispersion media, coatings, isotonic and absorption promoting or delaying agents, compatible with pharmaceutical administration and with the other components of the formulation. Such formulations can be contained in a tablet (coated or uncoated), capsule (hard or soft), microbead, emulsion, powder, granule, crystal, suspension, syrup or elixir.

Pharmaceutical compositions can be formulated to be compatible with a particular route of administration. Compositions for parenteral, intradermal, or subcutaneous administration can include a sterile diluent, such as water, saline solution, fixed oils, polyethylene glycols, glycerine, propylene glycol or other synthetic solvents. The preparation may contain one or more preservatives to prevent microorganism growth (e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose).

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyetheylene glycol), and suitable mixtures thereof. Fluidity can be maintained, for example, by the use of a coating such as lecithin, or by the use of surfactants. Antibacterial and antifungal agents include, for example, parabens, chlorobutanol, phenol, ascorbic acid and thimerosal. Including an agent that delays absorption, for example, aluminum monostearate and gelatin can prolonged absorption of injectable compositions.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

Additional pharmaceutical formulations and delivery systems are known in the art and are applicable in the methods of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky, et al., Drug Delivery Systems, R. L. Juliano, ed., Oxford, N.Y. (1980), pp. 253-315).

The Gal-3BP and compositions used in accordance with the invention, including proteins, treatments, therapies, agents, drugs and pharmaceutical formulations can be packaged in dosage unit form for ease of administration and uniformity of dosage. “Dosage unit form” as used herein refers to physically discrete units suited as unitary dosages treatment; each unit contains a quantity of the composition in association with the carrier, excipient, diluent, or vehicle calculated to produce the desired treatment or therapeutic (e.g., beneficial) effect. The unit dosage forms will depend on a variety of factors including, but not necessarily limited to, the particular composition employed, the effect to be achieved, and the pharmacodynamics and pharmacogenomics of the subject to be treated.

The invention provides kits including compositions of the invention (e.g., Gal-3BP polypeptide sequence, etc.), combination compositions and pharmaceutical formulations thereof, packaged into suitable packaging material. Kits can be used in various methods.

A kit typically includes a label or packaging insert including a description of the components or instructions for use in vitro, in vivo, or ex vivo, of the components therein. A kit can contain a collection of such components, e.g., Gal-3BP polypeptide sequence, alone, or in combination with another therapeutically useful composition (e.g., anti-inflammatory drug or agent).

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a response, disorder, or disease, or adverse symptom, for which a kit component may be used. Labels or inserts can include instructions for the clinician or for a subject for using one or more of the kit components in a method, treatment protocol or therapeutic regimen, or use. Instructions can include dosage amounts, frequency or duration, and instructions for practicing any of the methods, treatment protocols or therapeutic regimes and uses set forth herein. Exemplary instructions include, instructions for treating an undesirable or aberrant immune response, immune disorder, inflammatory response, inflammation or autoimmune response, disorder or disease. Kits of the invention therefore can additionally include labels or instructions for practicing any of the methods of the invention described herein including treatment, or diagnostic and detection methods.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Invention kits can additionally include other components. Each component of the kit can be enclosed within an individual container and all of the various containers can be within a single package. Invention kits can be designed for cold storage. Invention kits can further be designed to contain Gal-3BP polypeptide sequences.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a”, “and,” and “the” include plural referents unless the context clearly indicates otherwise. Thus, for example, reference to “a Gal-3BP polypeptide sequence” includes a plurality of such Gal-3BP polypeptide sequences or subsequences thereof, and reference to “an Gal-3BP polypeptide activity or function” can include reference to one or more Gal-3BP polypeptide activities or functions, and so forth.

As used herein, numerical values are often presented in a range format throughout this document. The use of a range format is merely for convenience and brevity and should not be construed as an inflexible limitation on the scope of the invention. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 2-10 (e.g., amino acids) includes 2, 3, 4, 5, 6, 7, 8, 9, 10, as well as 1.1, 1.2, 1.3, 1.4, 1.5, etc., 2.1, 2.2, 2.3, 2.4, 2.5, etc., and any numerical range within such a ranges, such as 2-3, 2-4, 2-6, 3-6, 3-7, 4-8, 5-9, 5-10, etc. In a further example, reference to a range of 2-10, 10-20, 20-30, 30-40, 40-50, 50-60, 60-75, 75-100 includes any numerical value or range within or encompassing such values.

As also used herein a series of ranges are disclosed throughout this document. The use of a series of ranges includes combinations of the upper and lower ranges to provide another range. This construction applies regardless of the breadth of the range and in all contexts throughout this document. Thus, for example, reference to a series of ranges such as 2-10, 10-20, 20-30, 30-40, 40-50, 50-60, 60-75, 75-100, includes ranges such as 2-20, 2-30, 5-20, 5-30, 5-40, 5-50, 5-60, 10-30, 10-40, 10-50, and 20-40, 20-30, 20-40, 20-50, 30-50, 30-60, 30-100, and 40-60, 40-70, 40-100, 50-75, 50-100, 60-100, and so forth.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example includes a description of various materials and methods.

### Monocyte-Derived Macrophages

Monocytes were isolated from peripheral blood of human healthy donors. Monocyte purity as assessed by flow cytometry for CD14 (clone HCD14, BioLegend, San Diego, Calif.) was routinely >97%. Monocytes were cultured for six days with M-CSF (100 ng/ml, Peprotech, Rocky Hill, N.J.) in macrophage serum-free medium (Gibco, Carlsbad, Calif.) supplemented with nutridoma SP (Roche, Nutley, N.J.), and penicillin/streptomycin (Sigma Aldrich). After this period, cells displayed the expected macrophage-like shape and were all positive for macrophage markers like CD68 (clone Y1/82A, BD Biosciences) or CD11b (clone CR3, BD Biosciences).

### Measurement of IL-2, IL-6, IL-12 and IL-10

Measurement of cytokines was carried out with Ready set Go ELISA kits (eBioscience).

### Measurement of Gal-3BP Expression

Gal-3BP concentration in conditioned medium was measured with BenderMed system human s90K/Mac-2BP Platinum ELISA kit.

### Immunocytochemistry for Human NFAT

Cells were fixed in formaldehyde 4%, 10-20 min, room temperature. Cells were then permeabilized in chilled methanol, 5-10 min, and −20° C. in and washed extensively with PBS, then blocked using 5% donkey serum in PBS. Primary antibody (Rabbit anti-NFATc2/NFATp (1:50, ImmunoGlobe), diluted in PBS with goat serum 10%) was applied over night in 4° C. Biotin anti-rabbit antibodies (1:200 Molecular Probes) were incubated for 1 hour. Streptavidin, (Alexa Fluor® 594 conjugate*2 mg/mL*Cat. No. S-32356) detection 1:1000 for 1 hour. DAPI (1:1,000, Sigma Aldrich) for 5 min at room temperature.

### Immunocytochemistry for Mouse NFAT and P65

Cells were fixed in formaldehyde 4%, 10-20 min, room temperature. Cells were then permeabilized in chilled methanol, 5-10 min, and −20° C. in and washed extensively with PBS. Blocking buffer (triton ×100, donkey serum 5%). Rabbit anti-NFAT1 and anti-P65 (1:50, Cell Signaling, diluted in PBS with donkey serum 5%), Alexa Fluor 568 donkey anti-rabbit IgG (Cat. No. A10042, Invitrogen, 1:200-1:2000) 1 hour. DAPI (1:1,000, Sigma Aldrich) for 5 min at room temperature.

### RNA Expression Induced by Gal-3BP, Real-Time RT-PCR

Total RNA was isolated using columns including a DNAse-step followed by reverse transcription (all Qiagen, Valencia, Calif.). Real-time PCR on a Light Cycler 480 (Roche, Indianapolis, Ind.) was performed in duplicates using 18S^{rRNA} as a normalizing gene. Product specificity was assessed by melting curve analysis and a minus RT control. Primer sequences:

| Gene | Forward sequence | Reverse sequence |
|---|---|---|
| Gal- | AGCAGCCACACCCAGAAG | GAGGAGGCTCCACACGG |
| 3BP | | |
| 18S | CGCCGCTAGAGGTGAAATTC | TTGGCAAATGCTTTCGCTC |

### Animals

Wild-type (wt) C57B1/6 mice were from Jackson Labs (Bar Harbor, Me.) and CYCAP knock-out (KO) (Lgals3bp^{-/-}) mice were provided by Irv Weissman (Stanford).

### Statistical Analyses

Statistical analyses were performed using Prism (GraphPad, La Jolla, Calif.) and MedCalc (MedCalc, Mariakerke, Belgium). P values were two-tailed and P<0.05, 0.01, or 0.001 are reported. Confidence intervals were calculated at the 95% level. All data are presented as means±standard error (SEM).

### Example 2

This example describes studies showing that Gal-3BP induces IL-2 secretion.

Human blood monocytes were incubated with M-CSF (100 ng/ml) for 6 days to produce monocyte-derived macrophages (MO). These macrophages were incubated with interferon-γ (250 u/ml) for 1 day to produce M1 macrophages or with IL-4 (20 units/ml) to produce M2 macrophages or with PGE-2 (1 μM) to produce Mreg macrophages and then challenged with LPS (10 ng/ml) for another day or with Gal-3BP (10 μg/ml over 48 h).

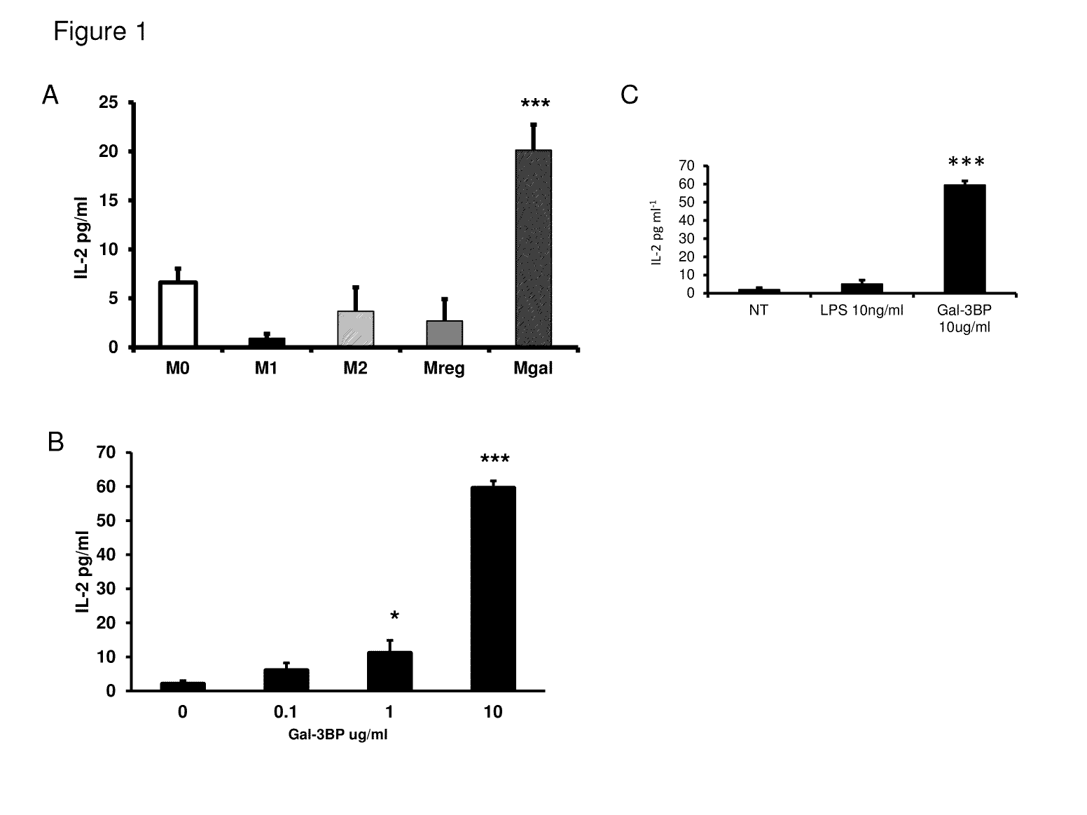

FIG. 1A shows that Gal-3BP induced production of IL-2 as measured by standard ELISA (***P=0.0003). FIG. 1B shows that Gal-3BP concentration dependently induces IL-2 production in human macrophages. Human blood monocytes were cultured as in (A) (*P=0.05, ***P<0.0001). These macrophages were incubated with Gal-3BP (0.1,1 and 10 μug/ml) for 6 hours and IL-2 was measured by ELISA. FIG. 1C shows that Gal-3BP but not LPS induces IL-2 production in human macrophages. Human blood monocytes were cultured as in (A). These macrophages were incubated with Gal-3BP (10 μg/ml) or LPS (10 ng/ml) for 6 hours (***P<0.0001).

### Example 3

This example shows studies indicating that Gal-3BP induces a unique macrophage phenotype, and that M1 macrophages produce Gal-3BP.

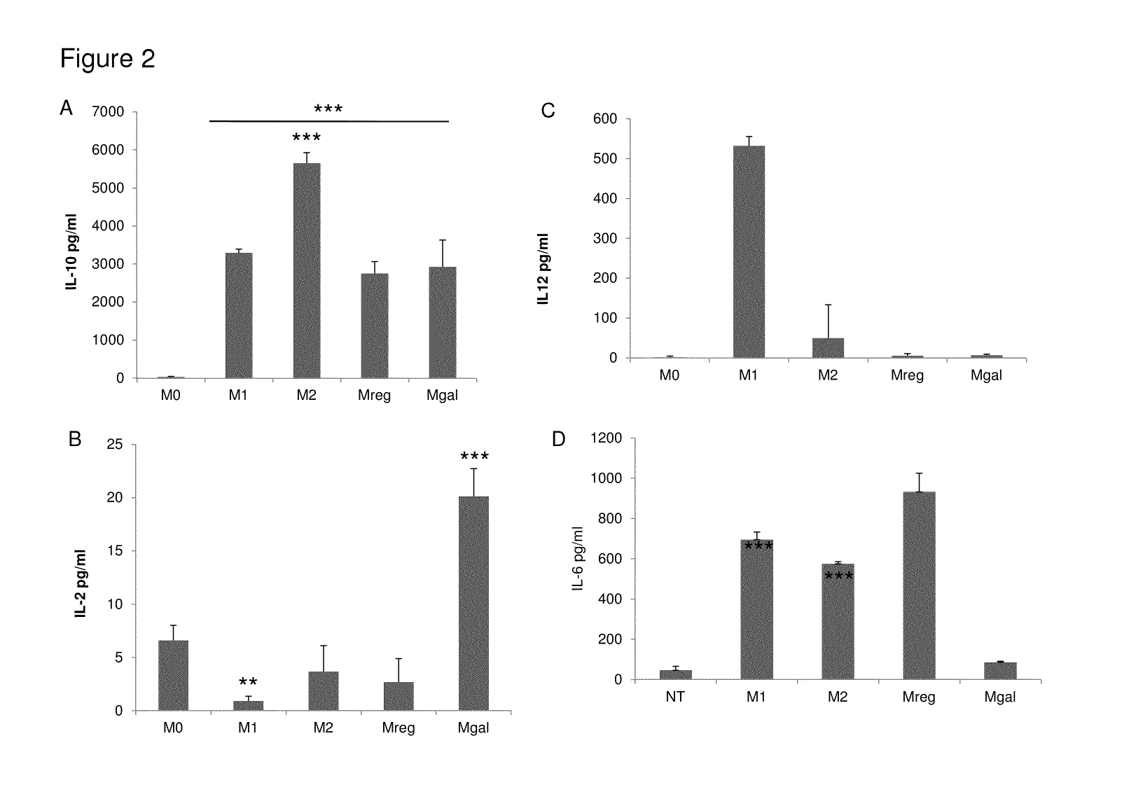

Human blood monocytes were incubated with M-CSF (100 ng/ml) for 6 days to produce monocyte-derived macrophages (M0). These macrophages were incubated with interferon-γ (250 units/ml) for 1 day to produce M1 macrophages or with IL-4 (20 units/ml) to produce M2 macrophages or with PGE-2 (1 μM) to produce Mreg macrophages and then challenged with LPS (10 ng/ml) for another day or with Gal-3BP (10 μg/ml over 48 h). Only Gal-3BP induced moderate IL-10 (typical of M2 and Mreg) (FIG. 2A); production of IL-2 (FIG. 2B), no IL-12 (typical of M1) (FIG. 2C); and little IL-6 (typical of M1) (FIG. 2D). P values are indicated on the graphs.

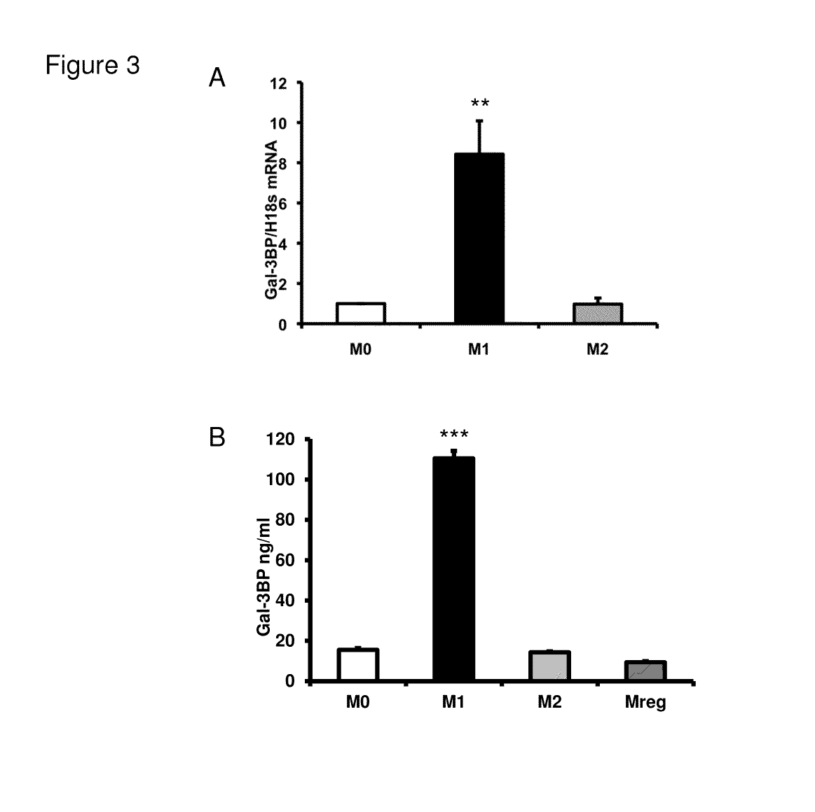

To show that M1 macrophages produce Gal-3BP, human blood monocytes were incubated with M-CSF (100 ng/ml) for 6 days to produce monocyte-derived macrophages (M0). These macrophages were incubated with interferon-γ (250 units/ml) for 1 day to produce M1 macrophages or with IL-4 (20 units/ml) to produce M2 macrophages or with PGE-2 (1 μM) to produce Mreg macrophages and then challenged with LPS (10 ng/ml). FIG. 3A shows Gal-3BP mRNA expression measured by quantitative RT-PCR, expressed relative to H18S (**P=0.0056 by paired t test, n=3 donors). FIG. 3B shows Gal-3BP protein production measured using ELISA (***P=0.0008). Only M1 macrophages induce Gal-3BP mRNA expression (FIG. 3A) and secrete significant amounts of Gal-3BP (FIG. 3B), although levels in the supernatant remain about 10 times lower than in blood. These studies demonstrate that M1 macrophages express Gal3-BP.

### Example 4

This example includes data showing that Gal-3BP triggers Ca²⁺ mobilization, and that Gal-3BP induces NFAT translocation to nucleus. This example also includes data indicating that NFAT is required for Gal-3BP-induced IL-2 secretion.

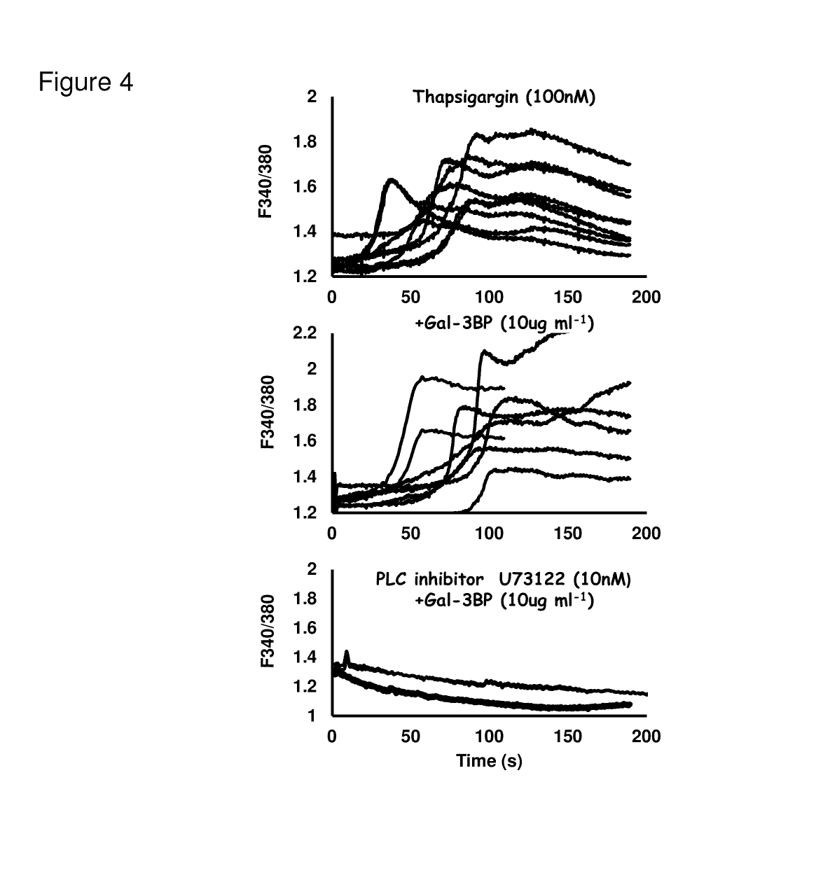

Human blood monocytes were cultured as described in Example 2 to produce MO macrophages, incubated with 1 μM fluo-4 for 30 minutes to quantify intracellular free Ca²⁺ concentrations, and then triggered with Gal-3BP (10 ug/ml) with or without the PLC-γ inhibitor U73122 (10 nM). Thapsigargin (100 nM) served as positive control. Ca²⁺ mobilization was assayed by fluorescence microscopy as previously described (Zanoni et al., Nature 460:264 (2009)). Each line is a measurement from a single cell (FIG. 4).

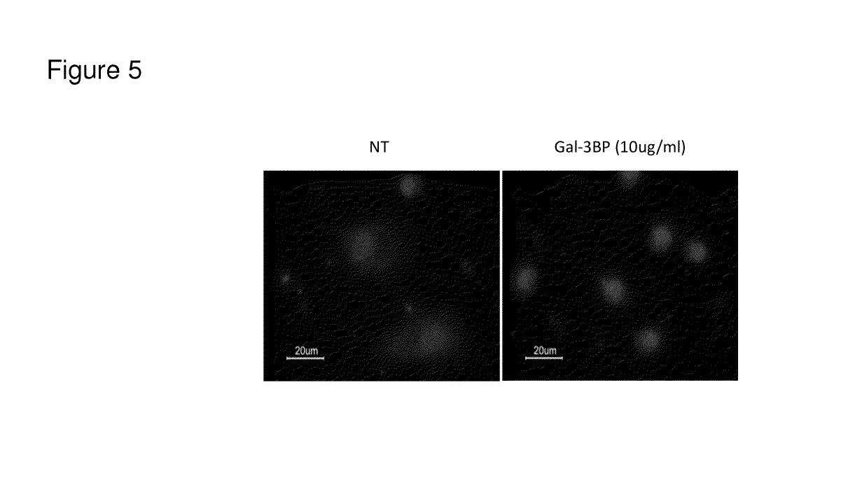

To determine if Gal-3BP induces NFAT translocation to nucleus, human blood monocytes were incubated with M-CSF (100 ng/ml) for 6 days to produce monocyte-derived macrophages (non-treated M0). These macrophages were then incubated with Gal-3BP (10 μg/ml for 2 hours). Both samples were stained with DAPI (nuclear stain) and an antibody to NFAT (anti-NFAT c2/p IG 209, affinity purified rabbit antibody, ImmunoGlobe). Nuclear translocation was assessed by immunofluorescence microscopy (FIG. 5).

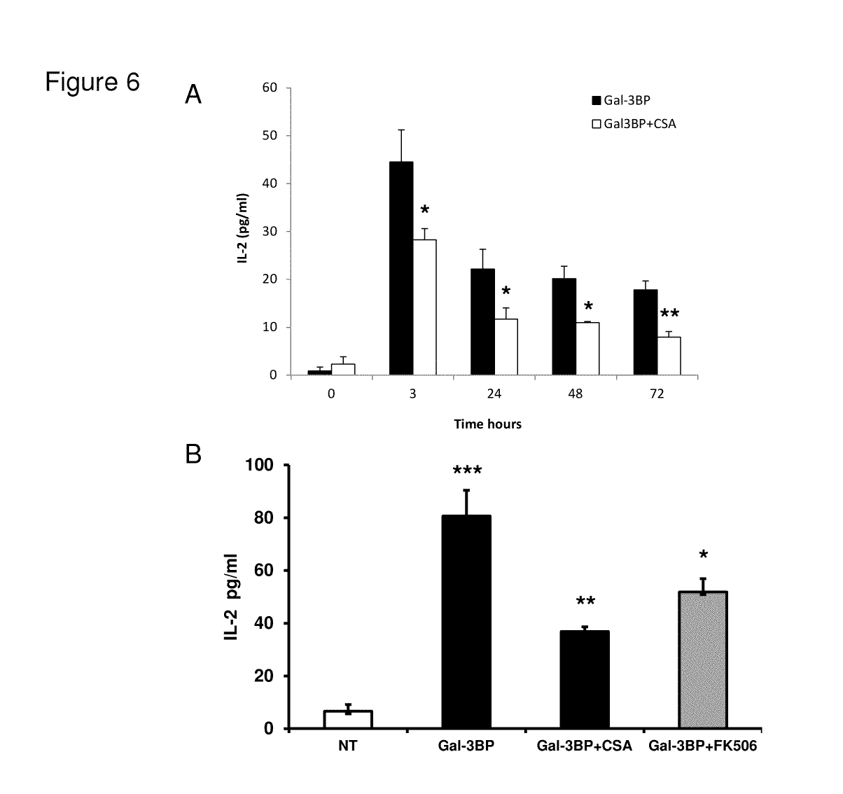

To determine if NFAT is required for Gal-3BP-induced IL-2 secretion, human blood monocytes were incubated with M-CSF (100 ng/ml) for 6 days to produce monocyte-derived macrophages (M0). Macrophages were incubated with Gal-3BP (10 μg/ml) for the times indicated, which induced secretion of IL-2 (FIG. 6A). IL-2 secretion was partially inhibited by the known NFAT inhibitor cyclosporine A (CSA 1 μM) (P values are 0.04, 0.04, 0.01 and 0.004, left to right respectively). Macrophages were alternatively treated with Gal-3BP (10 μg/ml) in the absence or presence of CSA (1 μM) or FK506 (1 μM), another known NFAT inhibitor (FIG. 6B). FK506 also inhibited IL-2 secretion by Gal-3BP (FIG. 6B). (P values are ***0.0004, **0.006 and *0.034)

The data demonstrate that NFAT is within the Gal-3BP signaling pathway and is required for Gal-3BP induced IL-2 secretion.

### Example 5

This example shows data indicating that Gal-3BP induces microRNA expression.

The targets of NFAT signaling are largely cytokines, growth factors and their receptors, proteins involved in cell-cell interactions, as well as microRNAs (Crabtree and Schreiber, Cell, 138: 210 (2009)). To evaluate Gal-3BP's ability to induce NFAT signaling, microRNA expression in human M0 macrophages following Gal-3BP treatment was measured.

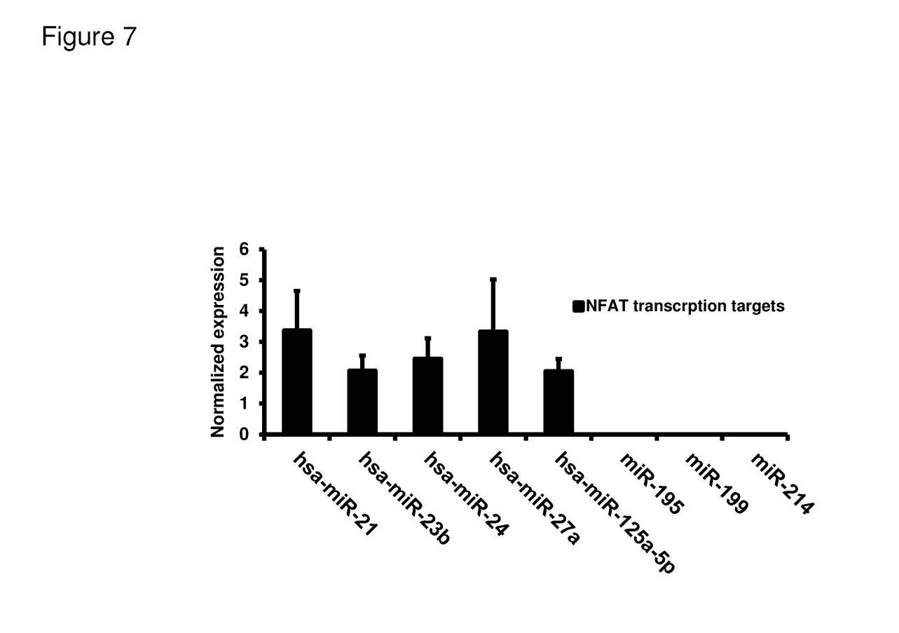

Human M0 macrophages were challenged with Gal-3BP (10 ug/ml), small RNAs were isolated and screened for microRNA expression using miRCURY™ Power Labeling Kit and hybridized on the miRCURY™ LNA Array (v.11.0). The samples were hybridized using a hybridization station and scanned using the Axon GenePix 4000B microarray scanner. GenePix pro V6.0 is used to extract the raw intensity of the image. Differentially expressed microRNAs were identified through fold-change screen. Out of eight microRNAs known to be transcriptional targets of NFAT, five were significantly up-regulated, including has-miR-21, has-miR-23b, has-miR-24, has-miR-27a, and has-miR-125a-5p (FIG. 7). These microRNAs reflect increased NFAT activity via Gal-3BP.

### Example 6

This example shows data indicating that dendritic cells from mice deficient in Gal-3BP fail to produce IL-2.

Mouse bone marrow derived dendritic cells (BMDC) obtained from femur and tibia of CYCAP knock-out (Lgals3bp^{-/-}) or WT mice were incubated for 6 days with GM-CSF (30 ng/ml). These BMDCs were incubated with interferon-γ (250 μg/ml) for 1 day to produce M1 phenotype (G1) or with IL-4 (20 ng/ml) to produce M2 phenotype (G2) or with PGE-2 (1 μM) to produce Mreg (Greg) phenotype and then challenged with LPS (10 ng/ml) for another day.

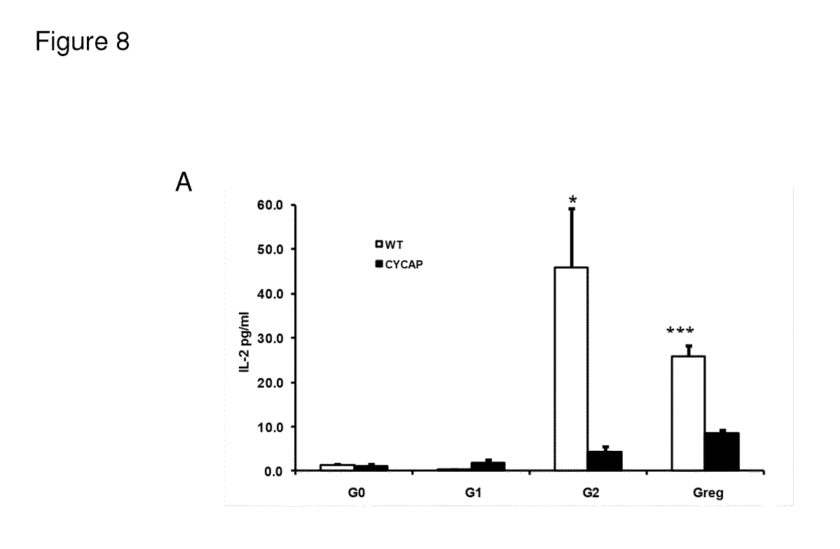

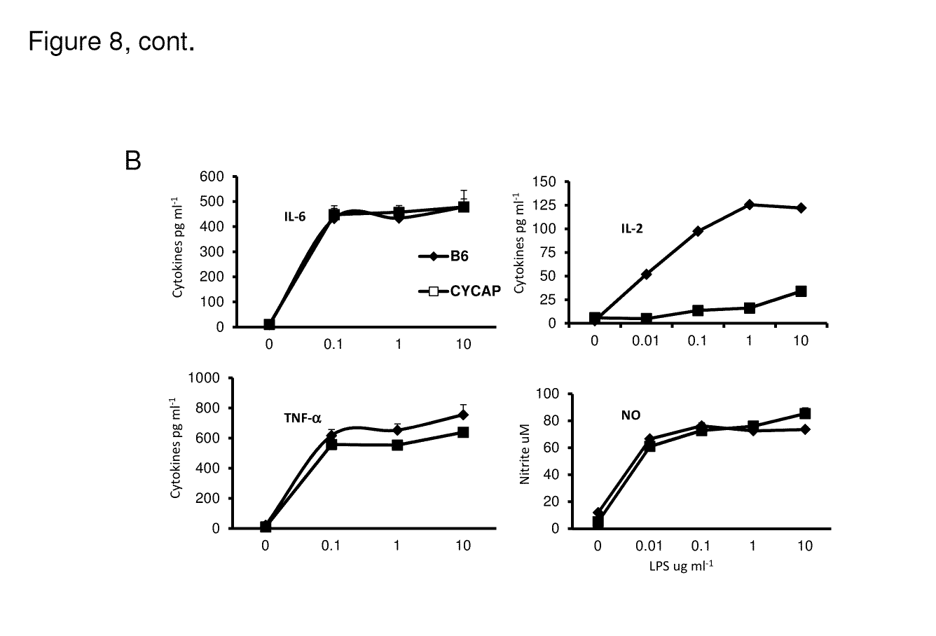

FIG. 8A shows IL-2 expression as measured by standard ELISA (P values are *0.02 and ***0.008, respectively). FIG. 8B shows that BMDCs from Gal-3BP deficient mice produce normal amount of inflammatory cytokines yet fail to produce IL-2. BMDCs from Gal-3BP deficient mice (CYCAP) or wild-type mice (B6) were triggered with LPS (0, 0.01, 0.1,11 and 10 ug/ml). Cytokines (IL-2, TNF and IL-6) were measured by ELISA and nitric oxide (NO) was measured using the Griess reagent.

These studies establish IL-2 as a robust readout for Gal3bp effects on myeloid derived cells, mouse dendritic cells or human macrophages. IL-2 is therefore a convenient biomarker.

### Example 7

This example shows data indicating that coupling LPS stimulation with Ca2+ mobilization restores IL-2 production, and that p65 nuclear translocation is intact in Gal-3BP knock-out mouse dendritic cells.

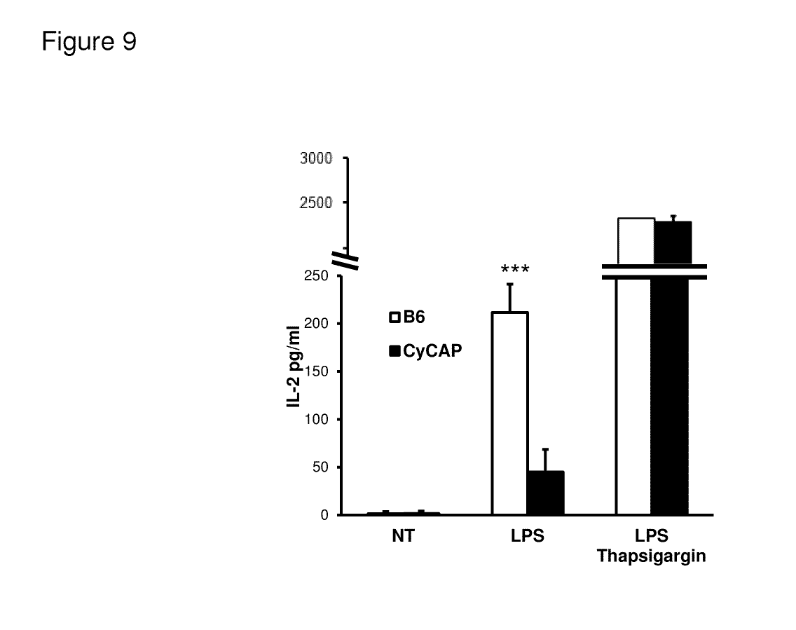

BMDC obtained from Gal-3BP deficient mice (CYCAP) or wild-type mice (B6) were triggered with LPS (100 ng/ml) in the presence or absence or thapsigargin (100 nM). FIG. 9 shows IL-2 production, as measured by ELISA. (***P<0.0001)

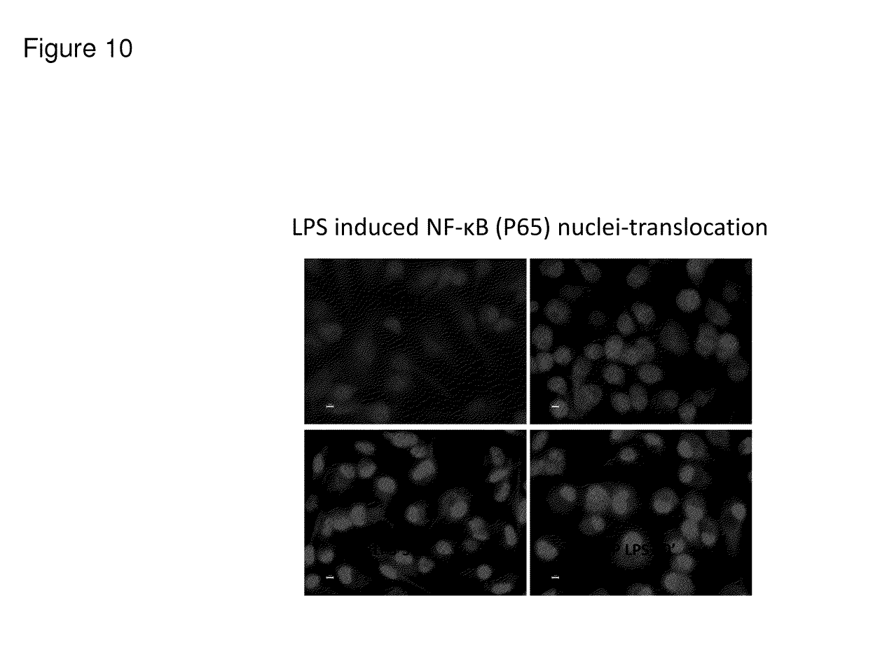

BMDC from Gal-3BP deficient mice (CYCAP) or wild-type mice (B6) were triggered with LPS (100 ng/ml) and stained with DAPI (nuclear stain) and an antibody to NF-kB (p65) (clone C22B4, Cell Signaling Technology). NFkB is the major inflammatory transcription factor complex and regulates hundreds of proinflammatory genes. Nuclear translocation of p65 was assessed by immunoflorescence microscopy (FIG. 10), and found to be intact in Gal-3BP deficient cells.

### Example 8

This example shows data indicating that Gal-3BP limits LPS-induced inflammatory cytokine production in mouse splenocytes.

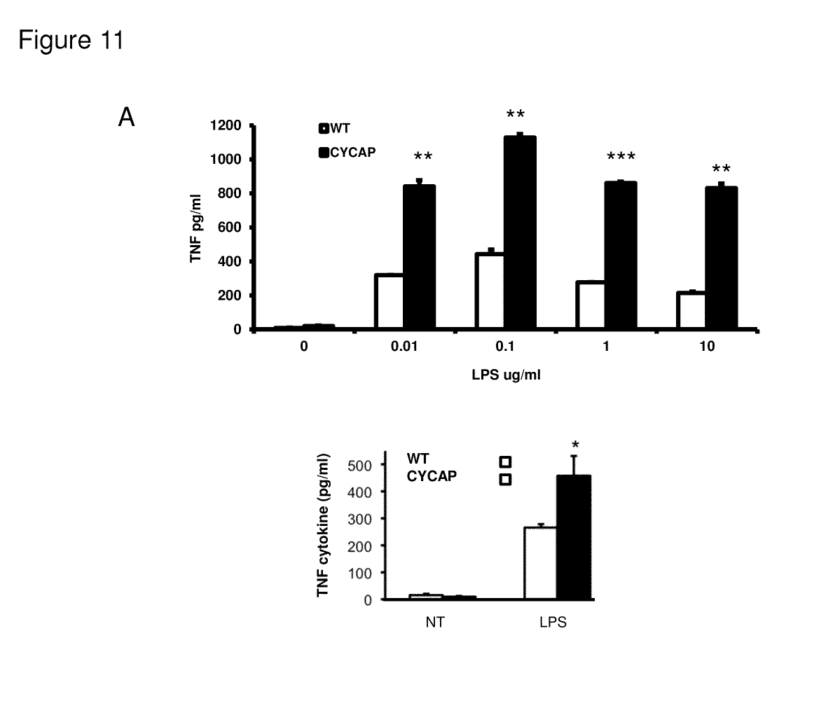

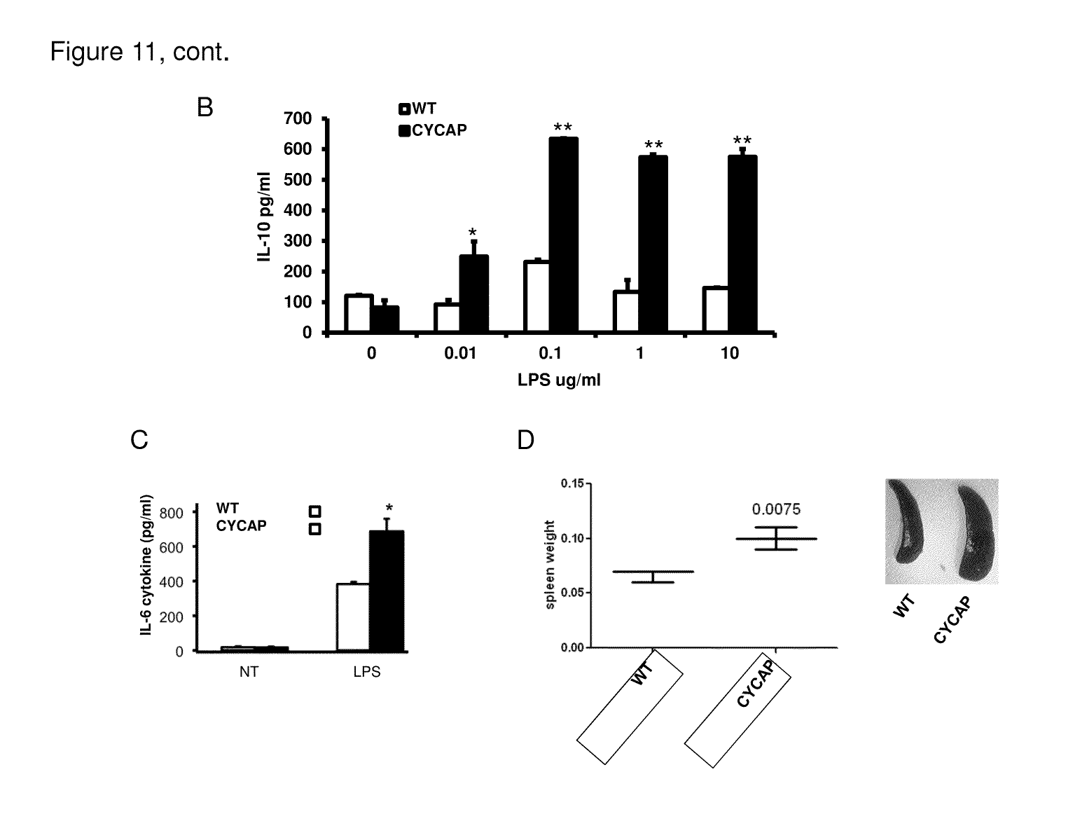

Splenocytes from mice deficient in Gal-3BP show robust cytokine production following LPS stimulation. Mouse splenocytes obtained from Gal-3BP deficient mice (CYCAP) or wild-type mice (WT) were incubated for 1 day in complete RPMI and then challenged with LPS (0.01-10 μg/ml) in the top panel of FIGS. 11A and 11B, or 0.1 μg/ml of LPS in the bottom panel of FIGS. 11A and 11C. Production of pro-inflammatory cytokines TNF-α (FIG. 11A) and IL-6 (FIG. 11C), and anti-inflammatory cytokine IL-10 (FIG. 11B) was measured by standard ELISA. For TNF-α production, P values are 0.002, 0.001, 0.0001 and 0.001, left to right respectively (top panel), and P=0.02 (lower panel). For IL-10 production, P values are 0.05, 0.0002, 0.003 and 0.003, left to right respectively. For IL-6 production, P=0.01. FIG. 11D shows spleen weight and appearance compared between Gal-3BP deficient mice (CYCAP) (n=5) and wild-type mice (WT) (n=5). (P value is 0.0075)

These studies shows that endogenous Gal-3BP normally dampens inflammatory cytokine output. When Gal-3BP is knocked out, there is an overshoot of baseline inflammation. Spleen size is correlated to inflammatory status, and expansion of leukocytes sub-population.

1. A method of decreasing, reducing, inhibiting, suppressing, limiting or controlling an undesirable or aberrant immune response, immune disorder, inflammatory response, inflammation or an autoimmune response, disorder or disease in a subject, comprising administering a Galectin-3 binding protein (Gal-3BP, BTBD17B) polypeptide or fragment thereof to a subject in an amount to decrease, reduce, inhibit, suppress, limit or control the undesirable or aberrant immune response, immune disorder, inflammatory response, inflammation or autoimmune response, disorder or disease in the subject, wherein the subject has not been diagnosed for an adverse cardiovascular event or cardiovascular disease.

2. (canceled)

3. The method of claim 1, wherein the treatment decreases, reduces, inhibits, suppresses, limits or controls an adverse symptom of the undesirable or aberrant immune response, immune disorder, inflammatory response, or inflammation, or an adverse symptom of the autoimmune response, disorder or disease.

4.-6. (canceled)

7. The method of claim 1, wherein the Galectin-3 binding protein (Gal-3BP, BTBD17B) polypeptide or fragment thereof stimulates, promotes, increases or induces interleukin-2 (IL-2) production by or secretion from macrophages or dendritic cells.

8. The method of claim 3, wherein the interleukin-2 (IL-2) stimulates, promotes, increases or induces survival, proliferation or differentiation of regulatory T cells (T regs).

9. The method of claim 1, wherein the Galectin-3 binding protein (Gal-3BP, BTBD17B) polypeptide or fragment thereof stimulates, promotes, increases or induces nuclear factor of activated T cells (NFAT) activation.

10. The method of claim 5, wherein the NFAT activation is indicated by stimulated, promoted, increased or induced macrophage microRNA expression, has-miR-21, has-miR-23b, has-miR-24, has-miR-27a or has-miR-125a-5p.

11. The method of claim 1, wherein the Galectin-3 binding protein (Gal-3BP, BTBD17B) polypeptide or fragment thereof decreases, reduces or inhibits TNF-alpha or interleukin-6 (IL-6) production or secretion by splenocytes or macrophages.

12.-13. (canceled)

14. The method of claim 1, wherein the subject has or has had an adverse symptom of the undesirable or aberrant immune response, immune disorder, inflammatory response, inflammation, or autoimmune response, disorder or disease, or has an existing undesirable or aberrant immune response, immune disorder, inflammatory response, inflammation, or autoimmune response, disorder or disease.

15. (canceled)

16. The method of claim 1, wherein the subject is in need of treatment for an undesirable or aberrant immune response, immune disorder, inflammatory response, inflammation, or autoimmune response, disorder or disease, or is at risk of an undesirable or aberrant immune response, immune disorder, inflammatory response, inflammation, or autoimmune response, disorder or disease.

17.-18. (canceled)

19. The method of claim 1, wherein the Galectin-3 binding protein (Gal-3BP, BTBD17B) polypeptide comprises a polypeptide fragment of full length Galectin-3 binding protein (Gal-3BP, BTBD17B) polypeptide.

20. The method of claim 1, wherein the Gal-3BP polypeptide is human, Pan troglodyte, Canis lupus familiaris, Bos Taurus, Mus musculus or Rattus norvegicus, SEQ ID NOs: 1-6, respectively.

21. (canceled)

22. The method of claim 1, wherein the Galectin-3 binding protein (Gal-3BP, BTBD17B) polypeptide comprises full length or a subsequence of:
```
                10 20 30 40 50 60
MTPPRLFWVW LLVAGTQGVN DGDMRLADGG ATNQGRVEIF YRGQWGTVCD NLWDLTDASV
                70 80 90 100 110 120
VCRALGFENA TQALGRAAFG QGSGPIMLDE VQCTGTEASL ADCKSLGWLK SNCRHERDAG
              130 140 150 160 170 180
VVCTNETRST HTLDLSRELS EALGQIFDSQ RGCDLSISVN VQGEDALGFC GHTVILTANL
              190 200 210 220 230 240
EAQALWKEPG SNVTMSVDAE CVPMVRDLLR YFYSRRIDIT LSSVKCFHKL ASAYGARQLQ
                250 260 270 280 290 300
GYCASLFAIL LPQDPSFQMP LDLYAYAVAT GDALLEKLCL QFLAWNFEAL TQAEAWPSVP
                        310 320 330 340 350 360
TDLLQLLLPR SDLAVPSELA LLKAVDTWSW GERASHEEVE GLVEKIRFPM MLPEELFELQ
                        370 380 390 400 410 420
FNLSLYWSHE ALFQKKTLQA LEFHTVPFQL LARYKGLNLT EDTYKPRIYT SPTWSAFVTD
                        430 440 450 460 470 480
SSWSARKSQL VYQSRRGPLV KYSSDYFQAP SDYRYYPYQS FQTPQHPSFL FQDKRVSWSL
                        490 500 510 520 530 540
VYLPTIQSCW NYGFSCSSDE LPVLGLTKSG GSDRTIAYEN KALMLCEGLF VADVTDFEGW
                        550 560 570 580
KAAIPSALDT NSSKSTSSFP CPAGHFNGFR TVIRPFYLTN SSGVD
```

23. The method of claim 12, wherein the Galectin-3 binding protein (Gal-3BP, BTBD17B) polypeptide subsequence is residues 24-124 (SRCR domain); residues 153-221 (BTB domain); or residues 260-360 (BACK domain).

24. The method of claim 1, wherein the Galectin-3 binding protein (Gal-3BP, BTBD17B) polypeptide comprises a fusion polypeptide, or a chimeric polypeptide.

25. The method of claim 14, wherein the chimeric polypeptide comprises a Galectin-3 binding protein (Gal-3BP, BTBD17B)/Immunoglobulin fusion polypeptide.

26.-33. (canceled)

34. The method of claim 1, wherein the immune disorder, inflammatory response, inflammation, autoimmune response, disorder or disease, comprises rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (γ_{c}) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), or hyper IgE syndrome.

35.-37. (canceled)

38. The method of claim 1, wherein the subject is not in need of treatment or therapy for artherosclerotic plaque formation, elevated blood cholesterol or a cardiovascular disease, or has not been diagnosed for cancer, has not been treated for cancer, or is in remission from cancer.

39. (canceled)

40. The method of claim 1, wherein the subject is a mammal.

41. (canceled)

42. A pharmaceutical composition comprising Galectin-3 binding protein (Gal-BP, BTBD17B) and an anti-inflammatory drug or agent.

43. A method of stimulating, promoting, increasing or inducing interleukin-2 (IL-2) production by or secretion from macrophages or dendritic cells or decreasing, reducing or inhibiting TNF-alpha or IL-6 production or secretion by splenocytes or macrophages, comprising administering Galectin-3 binding protein (Gal-3BP, BTBD17B) polypeptide or fragment thereof to a subject in an amount that stimulates, promotes, increases or induces interleukin-2 (IL-2) production by or secretion from macrophages or dendritic cells or decreases, reduces or inhibits TNF-alpha or IL-6 production or secretion by splenocytes or macrophages, wherein the subject has not been diagnosed for an adverse cardiovascular event or cardiovascular disease.

44. A method of decreasing, reducing or inhibiting TNF-alpha or IL-6 production or secretion by splenocytes or macrophages, comprising administering Galectin-3 binding protein (Gal-3BP, BTBD17B) polypeptide or fragment thereof to a subject in an amount that decreases, reduces or inhibits TNF-alpha or IL-6 production or secretion by splenocytes or macrophages, wherein the subject has not been diagnosed for an adverse cardiovascular event or cardiovascular disease.


SEQUENCE LISTING (10 sequences)

SEQ ID NO: 1 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 2 - protein, 585 aa, Pan troglodytes
MTPPRLFWVWLLVAGTQGVNDGDMRLADGGATNQGRVEIFYRGQWGTVCDNLWDLTDASVVCRALGFENATQALGRAAFGQGSGPIMLDEVQCMGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPRDPSFQTPLDLYAYAVATGDALLEKLCLQFLAWNFEALTQAEAWPSVPTDLLQLLLPRSDLAVPSELALLKAVDTWSWGERASHEEVEDLVEKIRFPMMLPEELFELQFNLSLYWSHEALFQKKTLQALEFHTVPFQLLARYKGLNLTEDTYKPRIYTSPTWSASVTDSSWSARKSQLVYQSRRGPLVKYSSNYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDINSSKSTSSFPCPAGHFNGFRTVIRPFYLTNSSGVD

SEQ ID NO: 3 - protein, 559 aa, Canis lupus famiilaris
MALPLVLWMCLLVAGTQGVKDGDMRLANGDTANEGRVEIFYSGRWGTVCDNLWDLMDASVVCRALGFENATEALGGAAFGPGKGPIMLDEVECTGTEPSLANCTSLGWMKSNCRHNQDAGVVCSNETRGAHTLDLSGELPAALEQIFDSQRGCDLSIRVKVKDQEEEGPHFCAHRLILAANPEAQALCKAPGSTVTMEVDAECLPVVRDFIRYLYSRRLDISLTSVKCFHKLASAYEAQQLQSFCASLFAILLPEDPSFQAPLDLYAYALATQDPVLEELCVQFLAWNFEGLTQATAWPRVPTALLQLLLSRSELAVPSELALLTALDVWSQERRPSHGEVARLVDKVRFPMMLPEHLFELQFNLSLYWSHEALFQKKILQALEFHTVPFRLLAQHRGLNLTEDAYQPRLYTSPTWSASVSRSSSRYWNYPYQSFQTPQHPSFLFQNKYISWSLVYLPTVQSCWNYGFSCSSDEVPLLGLSKSDYSDPTIGYENKALMRCGGRFVADVTDFEGQKALIPSALGTNSSRRPSLFPCLGGSFSSFQVVIRPFYLTNSSDVD

SEQ ID NO: 4 - protein, 555 aa, Bos taurus
MAPLRLFWIWLLVVGTRGVKDGDMRLADGGSANQGRVEIYYNGQWGTVCENMWDLTDASVVCRALGFQNATEALGGAAFGPGYGPIMLDEVRCTGTEPSLANCSSLGWMRSNCRHDKDASVICTNETRGVYTLDLSGELPAALEQIFESQKGCDLFITVKVREEDEIAMCAHKLILSTNPEAHGLWKEPGSRVTMEVDAECVPVVKDFIRYLYSRRIDVSLSSVKCLHKFASAYQAKQLQSYCGHLFAILIPQDPSFWTPLELYAYALATRDTVLEEICVQFLAWNFGALTQAEAWPSVPPALLQGLLSRTELVVPSELVLLLAVDKWSQERHTSHKEVEALVGQVRFPMMPPQDLFSLQFNLSLYWSHEALFQKKILQALEFHTVPFELLAQYWGLNLTEGTYQPRLYTSPTWSQSVMSSSYNPSRSFQTPQHPSFLFHDSSVSWSFVYLPTLQSCWNYGFSCSSDDPPLLALSKSSYSKSNPTIGYENRALLHCEGSFVVDVIDFKGWKALVPSALATNSSRSTSLFPCPSGVFSRFQVVIRPFYLTNSTDMD

SEQ ID NO: 5 - protein, 577 aa, Mus musculus
MALLWLLSVFLLVPGTQGTEDGDMRLVNGASANEGRVEIFYRGRWGTVCDNLWNLLDAHVVCRALGYENATQALGRAAFGPGKGPIMLDEVECTGTESSLASCRSLGWMVSRCGHEKDAGVVCSNDTTGLHILDLSGELSDALGQIFDSQQGCDLFIQVTGQGYEDLSLCAHTLILRTNPEAQALWQVVGSSVIMRVDAECMPVVRDFLRYFYSRRIEVSMSSVKCLHKLASAYGATELQDYCGRLFATLLPQDPTFHTPLDLYAYARATGDSMLEDLCVQFLAWNFEPLTQSESWSAVPTTLIQALLPKSELAVSSELDLLKAVDQWSTETIASHEDIERLVEQVRFPMMLPQELFELQFNLSLYQDHQALFQRKTMQALEFHTVPVEVLAKYKGLNLTEDTYKPRLYTSSTWSSLVMASTWRAQRYEYNRYNQLYTYGYGSVARYNSYQSFQTPQHPSFLFKDKQISWSATYLPTMQSCWNYGFSCTSNELPVLGLTTSSYSNPTIGYENRVLILCGGYSVVDVTSFEGSKAPIPTALDTNSSKTPSLFPCASGAFSSFRVVIRPFYLTNSTDMV

SEQ ID NO: 6 - protein, 574 aa, Ratus norvegicus
MALLWLLSVFLLVPGTQGAKDGDMRLVNGASASEGRVEIFYRGRWGTVCDNLWNLLDAHVVCRALGYENATQALSRAAFGPGKGPIMLDEVECTGNESSLANCSSLGWMVSHCGHEKDAGVVCSNDSRGIHILDLSGELPDALGQIFDSQQDCDLFIQVTGQGHGDLSLCAHTLILRTNPEAQALWQVVGSSVIMRVDAECMPVVRDFLRYFYSRRIEVSMSSVKCLHKLASAYGATELQGYCGRLFVTLLPQDPTFHTPLELYEYAQATGDSVLEDLCVQFLAWNFEPLTQAEAWLSVPNALIQALLPKSELAVSSELDLLKAVDQWSTATGASHGDVERLVEQIRFPMMLPQELFELQFNLSLYQGHQALFQRKTMEALEFHTVPLKVLAKYRSLNLTEDVYKPRLYTSSTWSSLLMAGAWSTQSYKYRQFYTYNYGSQSRYSSYQNFQTPQHPSFLFKDKLISWSATYLPTIQSCWNYGFSCTSDELPVLGLTTSSYSDPTIGYENKALILCGGYSVVDVTTFIGSKAPIPGTQETNSSKTPSLFPCASGAFSSFREVIRPFYLTNSTDTE

SEQ ID NO: 7 - DNA, 18 bp, Artificial Sequence
  Description of Artificial Sequence Forward Primer
agcagccacacccagaag

SEQ ID NO: 8 - DNA, 17 bp, Artificial Sequence
  Description of Artificial Sequence Reverse Primer
gaggaggctccacacgg

SEQ ID NO: 9 - DNA, 20 bp, Artificial Sequence
  Description of Artificial Sequence Forward Primer
cgccgctagaggtgaaattc

SEQ ID NO: 10 - DNA, 19 bp, Artificial Sequence
  Description of Artificial Sequence Reverse Primer
ttggcaaatgctttcgctc